(19) United States
(12) Patent Application Publication
(10) Pub. No.: US 2023/0060631 A1
(43) Pub. Date: Mar. 2, 2023

(54) DRY LIPOSOME ADJUVANT-CONTAINING VACCINES AND RELATED METHODS THEREOF

(71) Applicant: BOARD OF REGENTS, THE UNIVERSITY OF TEXAS SYSTEM, Austin, TX (US)

(72) Inventors: Zhengrong CUI, Austin, TX (US); Robert O. WILLIAMS, III, Austin, TX (US); Khaled ABOULFOTOUH, Austin, TX (US); Haiyue XU, Austin, TX (US)

(73) Assignee: BOARD OF REGENTS, THE UNIVERSITY OF TEXAS SYSTEM, Austin, TX (US)

(21) Appl. No.: 17/819,286

(22) Filed: Aug. 11, 2022

Related U.S. Application Data
(60) Provisional application No. 63/232,076, filed on Aug. 11, 2021.

(51) Int. Cl. A61K 9/70; A61K 9/127; A61K 39/39; A61K 9/00; A61K 39/25; C12N 7/00

(52) U.S. Cl. CPC A61K 9/7007; A61K 9/1271; A61K 39/39; A61K 9/0075; A61K 39/25; C12N 7/00; C12N 2710/16734; C12N 2710/16771; A61K 2039/55577

(57) ABSTRACT

Described herein are dry powder compositions of liposomes, liposomal adjuvant or liposomal adjuvanted vaccines. Formulations containing a cryoprotectant can be converted to dry powders using, e.g., thin-film freeze-drying (TFFD). The composition may comprise a liposomal adjuvant, such as AS01_{B} adjuvant, or also including an antigen, i.e., AS01_{B}-adjuvanted vaccine compositions.

The present application claims the benefit of priority to U.S. Provisional Application No. 63/232,076, filed on Aug. 11, 2021, the entire contents of which are hereby incorporated by reference.

### BACKGROUND

### I. Field

The disclosure generally relates to adjuvant compositions comprising liposomes, in particular those using AS01_{B} adjuvant systems. More particularly, the disclosure relates to thin film dry adjuvant compositions and vaccines produced from aqueous compositions.

### II. Related Art

Adjuvants are employed to enhance vaccines' immunogenicity (Di Pasquale et al., 2015). Adjuvant system 01_{B} (i.e., AS01_{B}) is among a series of adjuvant systems developed by GlaxoSmithKline Vaccines (Wavre, Belgium) to elicit durable humoral and cellular immune responses (Vandepapelire et al., 2008). AS01_{B} is a liposome formulation comprising two immunostimulants that work synergistically, namely 3-O-desacyl-4′-monophosphoryl lipid A (MPL) and QS-21, a highly purified triterpene glucoside (Didierlaurent et al., 2017). AS01_{B} is currently utilized as an adjuvant in the FDA approved SHINGRIX® (GlaxoSmithKline) vaccine for human use (i.e., Zoster Vaccine Recombinant, Adjuvanted, suspension for intramuscular injection). In this case AS01_{B} is provided as a suspension in a vial, which is used to reconstitute lyophilized varicella zoster virus glycoprotein E (gE) antigen in a separate vial immediately before administration.

The AS01_{B} suspension and lyophilized gE antigen should be stored refrigerated between 2° C. and 8° C. and should not be frozen. The vaccine should be discarded if the adjuvant suspension and/or the lyophilized antigen have been frozen or exposed to temperatures higher than 8° C. For example, it is recommended that after reconstitution, the SHINGRIX® vaccine should be administer immediately or stored refrigerated between 2° C. to 8° C. and used with 6 hours. It was reported that 14%-35% of refrigerators or shipments have exposed vaccines to freezing temperatures which could result in significant loss of freeze-sensitive vaccines (e.g., AS01_{B}-adjuvanted vaccines) (Matthias et al., 2007). As such, improved methods and formulations of AS01_{B}-adjuvanted vaccines that permit less stringent storage conditions and greater overall stability would be highly advantageous.

### SUMMARY

In an aspect there is provided a dry liposome powder composition, wherein said composition comprises monophosphoryl lipid A and/or saponin fraction QS-21 in a liposomal formulation, and said composition comprises a sugar or a sugar alcohol. The composition may further comprise an antigen, such as an antigen in a subunit vaccine. The liposomal formulation may comprise dioleoyl phosphatidylcholine and cholesterol. The composition may have a particle size distribution within 10%-50% of the range of a corresponding liquid adjuvant or adjuvanted vaccine composition. The sugar or sugar alcohol may be is present at about 40% to about 90% w/w or at about 70% w/w (i.e., 1% w/v to 8% w/v). The lipid to sugar/sugar alcohol ratio may be 1:4 w/w, 1:8 w/w, 1:15 w/w or 1:30 w/w. The antigen may be varicella zoster virus glycoprotein E, or comprises one, two, three or four distinct influenza hemagglutinin antigens. The liposomal formulation may comprise cholesterol and 1,2-dioleoyl-sn-glycero-3-phosphocholine, MPL, and QS-21.

The composition may comprise less than 5% water. The composition may further comprise an excipient, such as a salt, a buffer, a detergent, a polymer, an amino acid, or a preservative. Particular excipients may include one or more of disodium edetate, sodium chloride, sodium citrate, sodium succinate, sodium hydroxide, sodium alginate, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, chitosan, Formaldehyde, Dulbecco's modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer. The composition may comprise about 1-40% wt/wt of said excipient. The composition may be prepared from a liquid vaccine. The liposomal formulation may comprise one or more of a neutral lipid, a cationic lipid, an amphipathic lipid and/or an anionic lipid. The liposomal formulation may comprise a lipid selected from the group consisting of for example soya lecithin, cholesterol, Soya phosphatidylcholine, hydrogenated soybean phosphatidylcholine, 1,2-Distearoyl-sn-glycero-3-phosphoglycerol, phosphatidylcholine, dipalmitoylphosphatidylcholine, egg phosphatidylcholine, 1,2-diphytanoyl-sn-glycero-3-phosphocholine, 1,2-Dioleoyl-sn-glycero-3-phospho-rac-(1-glycerol), 1,2-dioleoyl-3-trimethylammonium-propane, 1,2-Dioleoyl-sn-glycero-3-phosphocholine, 1,2-dipalmitoyl-sn-glycero-3-phosphocholine, 1-paltnitoyl-2-lyso-sn-gycero-3-pllosplloclloline, 1-palmitoyl-2-oleoyl-sn-glycero-3-ethylphosphocholine, 1,2-dimyristoyl-sn-glycero-3-phosphocholine, 1-palmitoyl-2-oleoyl-sn-glycero-3-phosphocholine, and 1,2-dioleoyl-sn-glycero-3-phosphoethanolamine. The liposomes may contain MPL and/or QS-21, or neither.

Further provided is an adjuvant thin film made according to the methods as described herein. Also provided is a method for preparing an adjuvant thin film comprising applying a liquid adjuvant composition to a freezing surface, wherein said liquid adjuvant composition comprises monophosphoryl lipid A and/or saponin fraction QS-21 and a sugar or a sugar alcohol; and allowing said liquid adjuvant composition to disperse and freeze on said freezing surface thereby forming an adjuvant thin film. The composition may further comprise an antigen, such as an antigen in a subunit vaccine. The liquid adjuvant formulation may comprise dioleoyl phosphatidylcholine and cholesterol. The composition may have a particle size distribution upon reconstitution within 10-50% of the range of the liquid adjuvant composition. The sugar or sugar alcohol may be present at about 40% to about 90% w/w or about 70% w/w. The antigen may be varicella zoster virus glycoprotein E or comprises one, two, three or four distinct influenza hemagglutinin antigens. The vaccine thin film may comprise less than about 5% water and/or may further comprise an excipient, such as a salt, a buffer, a detergent, a polymer, an amino acid, preservative. The excipient may be one or more of disodium edetate, sodium chloride, sodium citrate, sodium succinate, sodium hydroxide, sodium alginate, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, chitosan, Formaldehyde, Dulbecco's modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer. The adjuvant thin film may comprise from about 1% to about 40% w/w of said excipient.

The liquid adjuvant composition may be exposed to said freezing surface from about 50 milliseconds to about 5 seconds. Exposure may comprise spraying or dripping droplets of said liquid vaccine or adjuvant composition. The freezing surface temperature may be about −180° C. to about 0° C., the diameters of the droplets are about 2-5 millimeters, and the droplets are dropped from a distance about 2 cm to 10 cm from the freezing surface. The method may comprise contacting the droplets with a freezing surface having a temperature differential of at least about 30° C. between the droplets and the surface. The freezing rate of said droplets may be between 10 K/second and 10³ K/second.

The method may further comprise removing the solvent from the adjuvant or adjuvanted vaccine thin film to form a dry adjuvant or adjuvanted vaccine composition. Such as wherein said removing of the solvent comprises lyophilization. The method may further comprise solvating said dry adjuvant composition, thereby forming a reconstituted liquid adjuvant formulation. The immunogenicity of said reconstituted liquid adjuvant formulation may be at least 80%, at least 90%, at least 95%, at least 98%, at least 99%, or 100% the immunogenicity of said liquid adjuvant composition. The reconstituted liquid adjuvant formulation may exhibit immunogenicity not significantly different from said liquid adjuvant composition. The immunogenicity of said reconstituted liquid adjuvant composition may be greater the immunogenicity of said liquid adjuvant composition. Solvating of said dry adjuvant composition may occur at least one year after preparing said dry adjuvant composition from said liquid adjuvant composition. Prior to said solvating of said dry adjuvant composition, said dry adjuvant or adjuvanted vaccine composition may be stored at about 4° C. for at least 99% of the time. Upon solvating said dry adjuvant or adjuvanted vaccine composition, the resulting reconstituted liquid adjuvant composition may remain homogeneous for at least one week and for up to 6 weeks when stored properly. Upon solvating said dry adjuvant or adjuvanted vaccine composition, the resulting reconstituted liquid adjuvant composition may not form a precipitate for at least one week when stored properly.

The liquid adjuvant composition or adjuvanted vaccine composition may comprise one or more of a neutral lipid, a cationic lipid, an amphipathic lipid and/or an anionic lipid, including one or more lipids selected from the group consisting of soya lecithin, cholesterol, Soya phosphatidylcholine, hydrogenated soybean phosphatidylcholine, 1,2-Distearoyl-sn-glycero-3-phosphoglycerol, phosphatidylcholine, dipalmitoylphosphatidylcholine, egg phosphatidylcholine, 1,2-diphytanoyl-sn-glycero-3-phosphocholine, 1,2-Dioleoyl-sn-glycero-3-phospho-rac-(1-glycerol), 1,2-dioleoyl-3-trimethylammonium-propane, 1,2-Dioleoyl-sn-glycero-3-phosphocholine, 1,2-dipalmitoyl-sn-glycero-3-phosphocholine, 1-paltnitoyl-2-lyso-sn-gycero-3-pllosplloclloline, 1-palmitoyl-2-oleoyl-sn-glycero-3-ethylphosphocholine, 1,2-dimyristoyl-sn-glycero-3-phosphocholine, 1-palmitoyl-2-oleoyl-sn-glycero-3-phosphocholine, and 1,2-dioleoyl-sn-glycero-3-phosphoethanolamine. The liquid adjuvant composition may comprise a lipid selected from the group consisting of cholesterol and 1,2-dioleoyl-sn-glycero-3-phosphocholine. The liquid adjuvant composition may comprise a pharmacologically active ingredient such as QS-21, MPL, or lyophilized CpG oligos.

In another embodiment, there is provided a method of inducing an immune response in a subject in need thereof, said method comprising administering a therapeutically effective amount of a composition as described herein to said subject, or a solvated, rehydrated or reconstituted version thereof. The subject may have or maybe at risk of contracting diphtheria, tetanus, pertussis, influenza, pneumonia, otitis media, bacteremia, meningitis, hepatitis, cirrhosis, anthrax poisoning, rabies, warts, poliomyelitis, Japanese encephalitis, coronavirus diseases, cancer, such as Clostridium tetani, Streptococcus pneumonia, Hepatitis A, Hepatitis B, Haemophilus influenza, Corynebacterium diphtheria, Bordetella pertussis, Human papillomavirus, Bacillus anthracis, Rabies virus, Japanese encephalitis virus, SARS/MERS-associated Coronavirus or Poliovirus. The subject may at the risk of developing malignant tumors or have tumors already.

In yet another embodiment, there is provided a method of inducing an immune response in a subject in need thereof, said method comprising administering a therapeutically effective amount of a solvated or unsolvated adjuvant thin film made according to the method as described herein to said subject. The subject may have or may be at risk of contracting diphtheria, tetanus, pertussis, influenza, pneumonia, otitis media, bacteremia, meningitis, hepatitis, cirrhosis, anthrax poisoning, rabies, warts, poliomyelitis, Japanese encephalitis, Coronavirus diseases, cancer, such as Clostridium tetani, Streptococcus pneumonia, Hepatitis A, Hepatitis B, Haemophilus influenza, Corynebacterium diphtheria, Bordetella pertussis, Human papillomavirus, Bacillus anthracis, Rabies virus, Japanese encephalitis virus, SARS-associated coronavirus, or Poliovirus. The subject may at the risk of developing malignant tumors or have tumors already.

In an aspect is provided a method of treating a disease in a patient in need of such treatment, the method including administering a therapeutically effective amount of a dry vaccine as described herein (e.g., in an aspect, embodiment, example, table, figure, or claims) (e.g., a reconstituted liquid vaccine as described herein) to the patient.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-C. Effect of TFFD process and repeated freezing-and-thawing on particle size distribution of liposomal adjuvant. The particle size distribution (FIG. 1A) and zeta potential (FIG. 1B) of liquid AS01_{B} and AS01_{B} powder after rehydration were examined using dynamic light scattering (DLS). (FIG. 1C) Liquid AS01_{B} adjuvant and its dry powder prepared by TFFD were exposed to three consecutive cycles of freezing at −20° C. for 8 h and thawing at 4° C. for 16 h. At the end of the third cycle, the powder was reconstituted with milli-Q water and mean particle size was determined by DLS. **p<0.01, ***p<0.001, ns: not significant.

FIGS. 2A-C. Effect of TFFD process and repeated freezing-and-thawing on particle size distribution of AS01_{B}-adjuvanted OVA model vaccine (i.e., ASOV-3). The particle size distribution (FIG. 2A) and zeta potential of (FIG. 2B) the liquid model vaccine and model vaccine powder after rehydration were examined using DLS. (FIG. 2C) Liquid AS01_{B}-adjuvanted OVA model vaccine and its dry powder prepared by TFFD were exposed to three consecutive cycles of freezing at −20° C. for 8 h and thawing at 4° C. for 16 h. At the end of the third cycle, the powder was reconstituted with milli-Q water and its mean particle size was determined by DLS. *p<0.05, ***p<0.001, ns: not significant.

FIG. 3A-D. Effect of different sucrose concentrations on the particle size distribution of AS01_{B}-adjuvanted OVA model vaccine subjected to TFFD and reconstitution.

FIGS. 4A-D. Effect of different trehalose concentrations on the particle size distribution of AS01_{B}-adjuvanted OVA model vaccine subjected to TFFD and reconstitution.

FIGS. 5A-D. Effect of different mannitol concentrations on the particle size distribution of AS01_{B}-adjuvanted OVA model vaccine subjected to TFFD and reconstitution.

FIGS. 6A-E. Characterization of thin-film freeze-dried AS01_{B}-adjuvanted OVA model vaccine powder (i.e., ASOV-3). (FIG. 6A) The integrity of OVA antigen after TFFD was evaluated using SDS-PAGE. (FIG. 6B) The intrinsic fluorescence of the three tryptophan residues of OVA protein before and after the AS01_{B}-adjuvanted OVA vaccine was subjected to TFFD. Tryptophan intrinsic fluorescence spectra were recorded using a PTI Quanmaster spectrofluorometer (Photon Technology International, Santa Clara, Calif.) at an excitation wavelength of 295 nm and the emission spectrum was collected from 300 nm to 400 nm (Li et al., 2015). (FIGS. 6C-D) Representative transmission electron microscopic (TEM) images of the AS01_{B}-adjuvanted OVA vaccine before (FIG. 6C) and after (FIG. 6D) it was subjected to TFFD. The morphology of the liposomes was investigated using an FEI Tecnai Transmission Electron. One drop of either the liquid vaccine or after it was subjected to TFFD, and reconstitution was deposited on carbon-coated grids and then the grids were stained with uranyl acetate (1% w/v). (FIG. 6E) X-ray diffractograms of AS01_{B}-adjuvanted OVA vaccine, sucrose, or AS01_{B} adjuvant alone, all in PBS as acquired with a Rigaku R-Axis Spider X-ray diffractometer (Rigaku, Tokyo, Japan). The diffraction peaks observed in the X-ray diffractograms are related to sodium chloride in the PBS.

FIGS. 7A-F. Immunogenicity of AS01_{B}-adjuvanted OVA vaccine (i.e., ASOV-3) before and after it was subjected to TFFD. Female C57BU6 mice, 6-8 weeks, were immunized with two doses of either OVA solution, liquid AS01_{B}-adjuvanted OVA model vaccine (i.e., before TFFD) or AS01_{B}-adjuvanted OVA model vaccine powder prepared by TFFD and reconstituted in milli-Q water 21 days apart. Each mouse received 5 μg of OVA with or without 135 μg of AS01_{B} (i.e., 100 μg of DOPC, 25 μg of cholesterol, 5 μg of MPL and 5 μg of QS-21). At the end of the study, mice were euthanized, and serum samples were collected for the measurement of anti-OVA IgG, IgG1 and IgG2a levels (FIGS. 7A-C) using enzyme-linked immunosorbent assay (ELISA). Also, splenocytes were collected for cell proliferation and cytokine (i.e., IFN-γ and IL-4) release following stimulation with OVA (10 μg/mL) for 48 hours (FIGS. 7D-F). Proliferation index was calculated as the proliferation of cells stimulated with OVA divided by the proliferation of unstimulated cells. *p<0.05, **p<0.01, ***p<0.001, ****p<0.0001, ns: not significant.

FIGS. 8A-B. Shown are particle size distribution curves (FIG. 8A) and mean particle size and polydispersity index (PDI) values (FIG. 8B) of SHINGRIX® vaccine after the AS01_{B} diluent was added to the lyophilized gE antigen vial (i.e., liquid SHINGRIX® vaccine) and after the liquid vaccine was converted to dry powder by TFFD and then reconstituted in water. As a control, the same liquid SHINGRIX® vaccine was also subjected to conventional shelf freeze-drying (i.e., a vial containing the liquid vaccine was frozen in a −80° C. freezer and then water was removed by sublimation). In FIG. 8B, **p<0.01; ns means not significant, when compared to liquid SHINGRIX® vaccine.

FIG. 9. Representative TEM images of SHINGRIX® vaccine after it was subjected to single-vial thin-film freeze-drying and reconstitution.

FIGS. 10A-C. Thin-film freeze-drying of AS01_{B}-adjuvanted Fluzone Quadrivalent® vaccine. Fluzone Quadrivalent® is an FDA approved unadjuvanted, liquid vaccine for the prevention of influenza disease caused by influenza subtype A and subtype B viruses. It contains the hemagglutinin (HA) antigenic proteins of four influenza strains at a total concentration of 60 μg HA/0.5 mL. AS01_{B} was prepared in house and then added to the vaccine at a concentration equivalent to 50 μg/0.5 mL of MPL and QS-21, each. Sucrose at a concentration of 77.39% (w/w) (i.e., 4% w/v) was employed as an excipient. (FIG. 10C) AS01_{B}-adjuvanted Fluzone Quadrivalent®, after subjected to TFFD or liquid, respectively, was subjected to three consecutive cycles of freezing at −20° C. for 8 h and thawing at 4° C. for 16 h. ****p<0.001.

FIGS. 11A-C. Characterization of thin-film freeze-dried, AS01_{B}-adjuvanted Fluzone Quadrivalent® vaccine powder. The antigen integrity was investigated using SDS-PAGE (FIG. 11A) and the effect of TFFD on antigens' structures was investigated by scanning the tryptophan fluorescence intensity and wavelength of maximum tryptophan fluorescence (FIG. 11B). (FIG. 11C) The functionality of HA proteins in the vaccine was evaluated by a hemagglutination assay using chicken red blood cells as previously described (Mandon et al., 2020). HA titers were reported as the reciprocal of the last dilution where hemagglutination was observed (i.e., absence of chicken red blood cell precipitation) and were expressed in HA units (HAUs)/50 μL.

FIG. 12. The aerosol performance of AS01_{B}-adjuvanted OVA model vaccine dry powder incorporating sucrose as a stabilizer and was prepared using TFFD. Data are mean t SD (n=3) of the percentage of sucrose recovered in the capsule or deposited on the DPI device, the adapter, the induction port, stages 1-7, and the micro-orifice collector (MOC).

FIGS. 13A & 13B show particle size of AS01_{B}/OVA with different concentrations of mucoadhesive agents, (A) before and (B) after being subjected to TFFD.

FIG. 14 show the SDS-PAGE results of AS01_{B}/OVA with 0%, 1.9% to 3.7% of CMC before and after TFFD.

FIGS. 15A & 15B show in vitro mucoadhesion test. (A) The experimental setting. (B) The displacement distances of AS01_{B}/OVA powders prepared with 0%, 1.9%, or 3.7% CMC on 1.5% agar gel or 1.5% agar gel with 2% porcine mucin. Data are mean from n=3.

### DETAILED DESCRIPTION

Here, the inventors disclose dry powder compositions of a liposomal formulation comprising immunostimulant molecules (i.e., AS01_{B}) and AS01_{B}-adjuvanted vaccines. Formulations containing sucrose at a concentration of 4% w/v were converted to dry powders using thin-film freeze-drying (TFFD). AS01_{B} adjuvant alone or AS01_{B}-adjuvanted vaccines (i.e., OVA model vaccine, Fluzone Quadrivalent® vaccine, or SHINGRIX® vaccine) were dropped onto a cryogenically cooled surface to form frozen thin-films rapidly. The frozen films were then lyophilized to sublime water. The dry powders maintained the particle size distribution of the AS01_{B} adjuvant and AS01_{B}-adjuvanted model and commercially available vaccines after they were exposed to repeated freezing and thawing. This solves the major problem associated with the AS01_{B}-adjuvanted vaccine as discussed above, namely, that the AS01_{B} adjuvant is present in liquid form in one vial and the antigen is present in a separate vial in a lyophilized form, must be kept at 2° C.-8° C., and must not be frozen from the point of manufacture until the point of use. As mentioned above, refrigerators or shipments often expose vaccines to harmful freezing temperatures. Moreover, cold chain accounts for −80% of the cost of vaccination programs in the developing countries (Chen et al., 2011). In addition, the vaccine may be manufactured and distributed as a dry powder in a single vial which may not be adversely affected by cold chain failure or may avoid cold chain requirement. The powder can be reconstituted using a commonly used diluent (e.g., water for injection or normal saline).

The resulting TFFD AS01_{B}-adjuvanted vaccine dry powder formulation has a number of distinct advantages. For example, thin film freezing is an ultra-rapid freezing process (i.e., 100-1000 K/s) that can preserve the particle size distribution and immunogenicity of AS01_{B}-adjuvanted vaccines via accelerating the nucleation rate and the formation of small ice crystals. AS01_{B} adjuvant alone or AS01_{B}-adjuvanted vaccines are dropped onto a cryogenically cooled surface to form frozen thin-films within, for example, 50 ms to 3 s. The frozen films are then lyophilized. Other high-speed freezing methods may also be employed. Technologies with slower freezing rate (e.g., conventional shelf freeze-drying) may result in phase separation and thus damage of proteins (e.g., denaturation and/or aggregation) and/or aggregations of the liposomal AS01_{B} or AS01_{B}-adjuvanted vaccines.

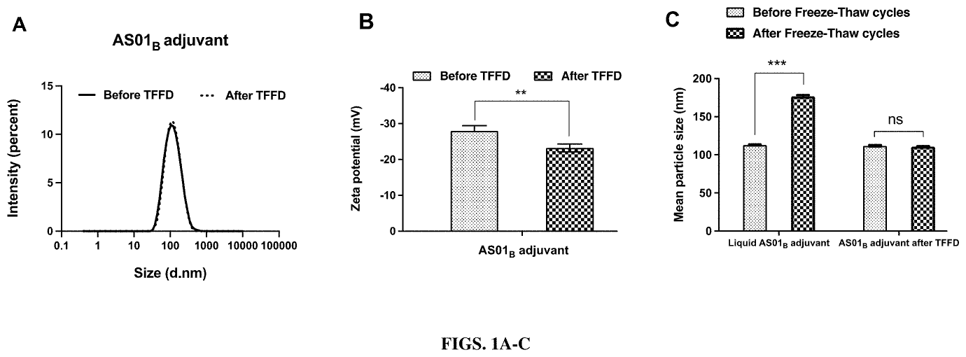

Moreover, the resultant dry powders of vaccines can be stored frozen, refrigerated or even at room temperature. Importantly, as shown in the examples below (see FIGS. 1A-C and 2C), the particle size distribution of a liquid AS01_{B} and AS01_{B}-adjuvanted model vaccine was maintained when subjected to repeated freeze-and-thaw. This is in contrast to the liquid vaccine formulations that showed significant aggregation upon repeated freeze-and-thaw. Thus, the materials described herein are unaffected by unintentional cold chain failure. This reduces vaccine loss and facilitates longer, less expensive and less risky distribution and storage of AS01_{B}-adjuvanted vaccines, and thus enhances all aspects of vaccination programs. In addition, the immunogenicity or antigen functionality of reconstituted dry powders of AS01_{B}-adjuvanted vaccine is equivalent to that of the liquid vaccine before subjected to thin-film freeze-drying. Also, a low concentration of cryoprotectant is employed (i.e., 4% w/v). The dry powder vaccines can also be administered via noninvasive routes (e.g., inhalation or intranasal) because thin-film freeze-dried powders usually have good aerosol properties due to the highly porous, brittle matrix structure of the dry powders.

These and other aspects of the disclosure are set out in detail below.

### I. Definitions

The abbreviations used herein have their conventional meaning within the chemical and biological arts. Description of compounds of the present disclosure is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions.

The terms “a” or “an,” as used in herein means one or more. In addition, the phrase “substituted with a[n],” as used herein, means the specified group may be substituted with one or more of any or all of the named substituents.

The terms “treating”, or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters, including the results of a physical examination, neuropsychiatric exams, and/or a psychiatric evaluation. For example, the certain methods presented herein successfully treat a disease associated with (e.g., caused by) an infectious agent (e.g., bacterium or virus). The term “treating” and conjugations thereof, include prevention of an injury, pathology, condition, or disease. The term “preventing” or “prevention” refers to any indicia of success in protecting a subject or patient (e.g., a subject or patient at risk of developing a disease or condition) from developing, contracting, or having a disease or condition (e.g., an infectious disease or diseases associated with an infectious agent), including preventing one or more symptoms of a disease or condition or diminishing the occurrence, severity, or duration of any symptoms of a disease or condition following administration of a prophylactic or preventative composition as described herein.

An “effective amount” is an amount sufficient for a composition (e.g., compound, vaccine, drug) to accomplish a stated purpose relative to the absence of the composition (e.g., compound, vaccine, drug) (e.g., achieve the effect for which it is administered, treat a disease (e.g., reverse or prevent or reduce severity), reduce spread of an infectious disease or agent, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a composition (vaccine) is an amount of a composition that, when administered to a subject, will have the intended prophylactic effect, e.g., preventing or delaying the onset (or reoccurrence) of an injury, disease (e.g., infectious disease), pathology or condition, or reducing the likelihood of the onset (or reoccurrence) of an injury, disease, pathology, or condition, or their symptoms. The full prophylactic effect does not necessarily occur by administration of one dose and may occur only after administration of a series of doses (e.g., prime-boost). Thus, a prophylactically effective amount may be administered in one or more administrations. The exact amounts will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of infection or one or more symptoms of infection in the absence of a composition (e.g., vaccine) as described herein (including embodiments).

“Contacting” is used in accordance with its plain ordinary meaning and refers to the process of allowing at least two distinct species (e.g., compositions, vaccines, bacterium, virus, biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a composition (e.g., vaccine) as described herein and a cell, virus, virus particle, protein, enzyme, or patient. In some embodiments contacting includes allowing a composition described herein to interact with a protein or enzyme that is involved in a signaling pathway. In some embodiments contacting includes allowing a composition described herein to interact with a component of a subject's immune system involved in developing immunity to a component of the composition.

As defined herein, the term “inhibition”, “inhibit”, “inhibiting” and the like in reference to a protein-inhibitor or interaction means negatively affecting (e.g., decreasing) the activity or function of the protein. In some embodiments, inhibition refers to reduction of a disease or symptoms of disease. In some embodiments, inhibition refers to reduction of the growth, proliferation, or spread of an infectious agent (e.g., bacterium or virus). In some embodiments, inhibition refers to preventing the infection of a subject by an infectious agent (e.g., bacterium or virus). In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating the signaling pathway or enzymatic activity or the amount of a protein.

The term “modulator” refers to a composition that increases or decreases the level of a target (e.g., molecule, cell, bacterium, virus particle, protein) or the function of a target or the physical state of the target.

The term “modulate” is used in accordance with its plain ordinary meaning and refers to the act of changing or varying one or more properties. “Modulation” refers to the process of changing or varying one or more properties. For example, as applied to the effects of a modulator on a target, to modulate means to change by increasing or decreasing a property or function of the target or the amount of the target.

“Patient” or “subject in need thereof” refers to a living organism suffering from or prone to a disease or condition that can be treated by administration of a composition (e.g., vaccine or pharmaceutical composition) as provided herein. Non-limiting examples include humans, other mammals, bovines, rats, mice, dogs, monkeys, goat, sheep, cows, deer, and other non-mammalian animals. In some embodiments, a patient is human. In some embodiments, a patient or subject in need thereof, refers to a living organism (e.g., human) at risk of developing, contracting, or having a disease or condition associated with an infectious agent (e.g., bacterium or virus).

“Disease” or “condition” refer to a state of being or health status of a patient or subject capable of being treated with the compositions (e.g., vaccines) or methods provided herein. In some embodiments, the disease is a disease related to (e.g., caused by) an infectious agent (e.g., bacterium or virus).

“Pharmaceutically acceptable excipient” and “pharmaceutically acceptable carrier” refer to a substance that aids the administration of an active agent to or absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure. In embodiments, an excipient is a salt, buffer, detergent, polymer, amino acid, or preservative. In embodiments, the excipient is disodium edetate, sodium chloride, sodium citrate, sodium succinate, sodium hydroxide, sodium alginate, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, chitosan, Formaldehyde, Dulbecco's modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer.

The term “preparation” is intended to include the formulation of the active compound with encapsulating material as a carrier providing a capsule in which the active component with or without other carriers, is surrounded by a carrier, which is thus in association with it.

As used herein, the term “administering” means oral administration, administration as a suppository, topical contact, intravenous, intraperitoneal, intramuscular, intralesional, intrathecal, intranasal, intradermal, mucosal, intrarectal, intravaginal, topical, transcutaneous, or subcutaneous administration. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example infection therapies such as antiviral drugs or a vaccine (e.g., different vaccine). The compositions (e.g., vaccines) of the disclosure can be administered alone or can be coadministered to the patient. Coadministration is meant to include simultaneous or sequential administration of the compounds individually or in combination (more than one composition) and includes vaccine administration in a prime-boost method. Thus, the preparations can also be combined, when desired, with other active substances (e.g., to reduce metabolic degradation, increase immune response (e.g., adjuvant)). The compositions of the present disclosure can be delivered by transdermally, by a topical route, transcutaneously, formulated as solutions, suspensions, emulsions, gels, creams, ointments, pastes, jellies, paints, powders, and aerosols.

The term “administer (or administering) a vaccine” means administering a composition that prevents or treats an infection in a subject. Administration may include, without being limited by mechanism, allowing sufficient time for the vaccine to induce an immune response in the subject or to reduce one or more symptoms of a disease.

The terms “peptide,” “polypeptide,” and “protein” are used interchangeably herein to refer to a polymer of amino acid residues. An “antigenic protein” is a protein that may be included in a vaccine as an antigen. In embodiments, an antigenic protein may be an antigenic protein conjugated to a sugar (i.e., saccharide) (e.g., monosaccharide, disaccharide, polysaccharide) “antigenic protein saccharide conjugate”. In embodiments, an antigenic protein may be an antigenic protein that is not conjugated to a sugar (saccharide).

The term “peptidyl” and “peptidyl moiety” means a monovalent peptide.

The term “amino acid” refers to naturally occurring and synthetic amino acids, as well as amino acid analogs. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. An oligomer comprising amino acid mimetics is a peptidomimetic. A peptidomimetic moiety is a monovalent peptidomimetic.

The term “isolated” refers to a nucleic acid, polynucleotide, polypeptide, protein, or other component that is partially or completely separated from components with which it is normally associated (other proteins, nucleic acids, cells, etc.). In some embodiments, an isolated polypeptide or protein is a recombinant polypeptide or protein.

The terms “dose” and “dosage” are used interchangeably herein. A dose refers to the amount of active ingredient given to an individual at each administration. For the present methods and compositions provided herein, the dose may generally refer to the amount of disease treatment. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition and depends on the route of administration. For example, a dosage form can be in a liquid form for nebulization, e.g., for inhalants, in a tablet or liquid, e.g., for oral delivery, or a saline solution, e.g., for injection.

The term “adjuvant” is used in accordance with its plain ordinary meaning within Immunology and refers to a substance that is commonly used as a component of a vaccine. Adjuvants may increase an antigen specific immune response in a subject when administered to the subject with one or more specific antigens as part of a vaccine. In some embodiments, an adjuvant accelerates an immune response to an antigen. In some embodiments, an adjuvant prolongs an immune response to an antigen. In some embodiments, an adjuvant enhances an immune response to an antigen. In some embodiments, an adjuvant is a liposomal adjuvant.

The terms “bind”, “bound”, “binding”, and other verb forms thereof are used in accordance with their plain ordinary meaning within Enzymology and Biochemistry and refer to the formation of one or more interactions or contacts between two compositions that may optionally interact. Binding may be intermolecular or intramolecular.

The term “vaccine” is used according to its plain ordinary meaning within medicine and Immunology and refers to a composition including an antigenic component (e.g., antigenic protein) for administration to a subject (e.g., human), which elicits an immune response to the antigenic component (e.g., antigen protein). In some embodiments a vaccine is a therapeutic. In some embodiments, a vaccine is prophylactic. In some embodiments a vaccine includes one or more adjuvants (e.g., a liposomal adjuvant). A liquid vaccine is a vaccine in liquid form, which may be for example a solution, suspension, emulsion, or dispersion or the antigenic component (e.g., antigenic protein) of the vaccine and may optionally include other components. A dry vaccine is a vaccine comprising 5% or less of water.

A vaccine is a preparation employed to improve immunity to a particular disease. Vaccines include an agent, which is used to induce a response from the immune system of the subject. Various agents that are typically used in a vaccine include, but are not limited to: killed, but previously virulent, micro-organisms; live, attenuated microorganisms; inactivated toxic compounds that are produced by microorganism that cause an illness; protein subunits of microorganisms; and conjugates. Examples of vaccines that may be converted into a powder vaccine according to the methods described herein include, but are not limited to: influenza vaccine, cholera vaccine, bubonic plague vaccine, polio vaccine, Hepatitis A vaccine, rabies vaccine, yellow fever vaccine, measles vaccine, rubella vaccine, mumps vaccine, typhoid vaccine, tuberculosis vaccine, tetanus vaccine, diphtheria vaccine, diphtheria-tetanus-pertussis vaccine, Hepatitis B vaccine, human papillomavirus (HPV) vaccine, Pneumococcal conjugate vaccines, influenza vaccine, botulism vaccine, polio vaccine, and anthrax vaccines.

The term “prime-boost” or “prime boost” as applied to a methodology of administering vaccines is used according to its plain ordinary meaning in Virology and Immunology and refers to a method of vaccine administration in which a first dose of a vaccine or vaccine component is administered to a subject or patient to begin the administration (prime) and at a later time (e.g., hours, days, weeks, months later) a second vaccine is administered to the same patient or subject (boost). The first and second vaccines may be the same or different but are intended to both elicit an immune response useful in treating or preventing the same disease or condition. In some embodiments the prime is one or more viral proteins or portions thereof and the boost is one or more viral proteins or portions thereof.

The term “associated” or “associated with” as used herein to describe a disease (e.g., a virus associated disease or bacteria associated disease) means that the disease is caused by, or a symptom of the disease is caused by, what is described as disease associated or what is described as associated with the disease. As used herein, what is described as being associated with a disease, if a causative agent, could be a target for treatment of the disease.

The term “vaccinate” or additional verb forms thereof refer to administering a vaccine to a subject (e.g., human) and eliciting an antigen specific immune response, wherein the antigen (e.g., antigenic protein) is included in the vaccine. The term “vaccinate” may also refer to eliciting an antigen specific immune response against an administered antigen (e.g., antigenic protein). In some embodiments, vaccinate is to provide prophylaxis against a disease or infectious agent.

The term “portion” refers to a subset of a whole, which may also be the whole. In some embodiments, a portion is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. In some embodiments, a portion is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. Unless indicated otherwise, the term “about” in the context of a numeric value indicates the nominal value t 10% of the nominal value. In some embodiments, “about” may be the nominal value.

### II. Compostions

In one aspect there is provided a dry powder of a liposome such as AS01_{B}, or a vaccine including an AS01_{B} adjuvant. AS01_{B} is present in an FDA approved human vaccine (i.e., SHINGRIX®). Its combination with recombinant VZV gE was found to significantly boost the antigen's immunogenicity during a vaccine development program by Glaxo Smith-Kline. The adjuvant suspension is composed of 3-O-desacyl-4′-monophosphoryl lipid A (MPL) from Salmonella minnesota and QS-21, a saponin fraction purified from plant extract of Quillaja saponaria Molina, combined in a liposomal formulation.

In embodiments, at least 60% of the antigenic protein is not denatured after a vaccine is subjected to TFFD. In embodiments, at least 70% of the antigenic protein is not denatured. In embodiments, at least 80% of the antigenic protein is not denatured. In embodiments, at least 90% of the antigenic protein is not denatured. In embodiments, at least 95% of the antigenic protein is not denatured. In embodiments, at least 60% of the antigenic protein is in a conformationally native state. In embodiments, at least 70% of the antigenic protein is in a conformationally native state. In embodiments, at least 80% of the antigenic protein is in a conformationally native state. In embodiments, at least 90% of the antigenic protein is in a conformationally native state. In embodiments, at least 95% of the antigenic protein is in a conformationally native state. A “conformationally native state” is a folded conformation corresponding to an operative or functional protein that can induce a specific immune response. A “denatured” protein is a protein having a conformation differing from the folded active or functional conformation of the protein, wherein the denatured protein has a reduced level of activity or function. In embodiments, the antigen protein is an unconjugated antigenic protein including virus-like particles (VLPs). In embodiments, the antigenic protein is an antigenic protein sugar (saccharide) conjugate. In embodiments, the sugar (saccharide) is a monosaccharide. In embodiments, the sugar (saccharide) is a disaccharide. In embodiments, the sugar (saccharide) is a polysaccharide.

In embodiments, the dry vaccine includes less than 5% water. In embodiments, the dry vaccine includes less than 4% water. In embodiments, the dry vaccine includes less than 3% water. In embodiments, the dry vaccine includes less than 2% water. In embodiments, the dry vaccine includes less than 1% water. In embodiments, the dry vaccine includes less than 5% water (wt/wt). In embodiments, the dry vaccine includes less than 4% water (wt/wt). In embodiments, the dry vaccine includes less than 3% water (wt/wt). In embodiments, the dry vaccine includes less than 2% water (wt/wt). In embodiments, the dry vaccine includes less than 1% water (wt/wt). In embodiments, the dry vaccine includes about 5% water. In embodiments, the dry vaccine includes about 4% water. In embodiments, the dry vaccine includes about 3% water. In embodiments, the dry vaccine includes about 2% water. In embodiments, the dry vaccine includes about 1% water. In embodiments, the dry vaccine includes about 5% water (wt/wt). In embodiments, the dry vaccine includes about 4% water (wt/wt). In embodiments, the dry vaccine includes about 3% water (wt/wt). In embodiments, the dry vaccine includes about 2% water (wt/wt). In embodiments, the dry vaccine includes about 1% water (wt/wt). In embodiments, the dry vaccine includes less than 5% water (v/v). In embodiments, the dry vaccine includes less than 4% water (v/v). In embodiments, the dry vaccine includes less than 3% water (v/v). In embodiments, the dry vaccine includes less than 2% water (v/v). In embodiments, the dry vaccine includes less than 1% water (v/v). In embodiments, the dry vaccine includes about 5% water (v/v). In embodiments, the dry vaccine includes about 4% water (v/v). In embodiments, the dry vaccine includes about 3% water (v/v). In embodiments, the dry vaccine includes about 2% water (v/v). In embodiments, the dry vaccine includes about 1% water (v/v).

In embodiments, the dry vaccine includes an excipient. In embodiments, the dry vaccine includes a plurality of different excipients. In embodiments, the excipient is a salt, buffer, detergent, polymer, amino acid, or preservative. In embodiments, the excipient is disodium edetate, sodium chloride, sodium citrate, sodium succinate, sodium hydroxide, sodium alginate, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, chitosan, Formaldehyde, Dulbecco's modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer. In embodiments, the excipient is trehalose. In embodiments, the dry vaccine includes about 60% or above, wt/wt, of the excipient. In embodiments, the dry vaccine includes less than 4% wt/wt of the excipient. In embodiments, the dry vaccine includes less than 3% wt/wt of the excipient. In embodiments, the dry vaccine includes less than 2% wt/wt of the excipient. In embodiments, the dry vaccine includes less than 1% wt/wt of the excipient. In embodiments, the dry vaccine includes less than 0.5% wt/wt of the excipient. In embodiments, the dry vaccine includes about 5% wt/wt of the excipient. In embodiments, the dry vaccine includes about 4% wt/wt of the excipient. In embodiments, the dry vaccine includes about 3% wt/wt of the excipient. In embodiments, the dry vaccine includes about 2% wt/wt of the excipient. In embodiments, the dry vaccine includes about 1% wt/wt of the excipient. In embodiments, the dry vaccine includes about 0.5% wt/wt of the excipient. In some embodiments, the dry vaccine may comprise from about 0.5%, 1%, 2%, 3%, 4%, 5%, 6%, 7%, 8%, 9%, 10%, 12%, 14%, 16%, 18%, 20%, 25%, 30%, 35%, 40%, 50%, to about 60% wt/wt, or any range derivable therein.

In embodiments, the dry vaccine includes liposomal particles. In embodiments, the dry vaccine is prepared from a liquid vaccine.

In an embodiment, a powder (e.g., dry) vaccine, which retains its efficacy, may be made from a vaccine composition. The method includes obtaining a liquid (e.g., aqueous) vaccine composition. The vaccine composition includes an agent that resembles a disease-causing microorganism, or a compound associated with the disease-causing microorganism (e.g., antigenic protein). The vaccine composition also includes an adjuvant (e.g., liposomal adjuvant). The vaccine composition is frozen to obtain a frozen vaccine composition (e.g., vaccine thin film). Water is removed from the frozen vaccine composition to form a powder (e.g., dry) vaccine that includes the agent or compound (e.g., antigenic protein) and the adjuvant (e.g., liposomal adjuvant).

A cryoprotectant may be added to the vaccine composition to protect the organisms or agents present in the composition (either live or dead) from damage during the freezing process. Examples of cryoprotectants include glycerol, monosaccharides, and polysaccharides (e.g., trehalose), polymers (e.g., PVP), amino acids (e.g., leucine), or proteins (e.g., human serum albumin). A cryoprotectant may be present in amounts up to about 90% by weight in the dry vaccine powder.

Additionally, the solid form of the vaccine is expected to be advantageous over vaccine dispersion (i.e., suspension) for stockpiling vaccines that are critical to national security and public health. For example, botulism is a life-threatening disease caused by botulinum neurotoxins (BoNTs), which are produced by one of the seven structurally similar Clostridium botulinum serotypes, designated A to G. Each of the toxins is immunologically distinct, except that serotypes C and D share significant cross-homology. BoNTs are the most poisonous substances known in nature. A single gram of crystalline toxin, evenly dispersed and inhaled, would kill more than one million people. Previously, an investigational pentavalent botulism toxoid (PBT) vaccine aiming to protect against BoNT serotypes A-E had been available. However, as of November 2011, the PBT vaccine has been discontinued by the Centers for Disease Control and Prevention (CDC), based on “an assessment of the available data, which indicate a decline in immunogenicity of some of the toxin serotypes”. Since the investigative PBT vaccine was the only botulism vaccine available in the U.S., discontinuation of it has significant national security implications.

In another embodiment, an aqueous vaccine composition may be composed of an agent that forms particles having a particle size of less than about 200 nm (e.g., less than 10, 20, 30, 40, 50, 60, 70, 80, 90, 100, 110, 120, 130, 140, 150, 160, 170, 180, 190, 200 nm). In some embodiments, particles having a diameter of less than 200 nm (e.g., less than 10, 20, 30, 40, 50, 60, 70, 80, 90, 100, 110, 120, 130, 140, 150, 160, 170, 180, 190, 200 nm) may be used as adjuvants in a vaccine composition. The aqueous vaccine composition may be used to vaccinate a subject against the disease related to the agent. In some embodiments, the aqueous vaccine composition can be converted to a vaccine powder, as described above, for storage, for use as an inhalant, or use in other delivery modes.

In embodiments, a dry vaccine is the dry vaccine described herein, including in embodiments, examples, tables, figures, and claims. In embodiments, a dry vaccine is a dry vaccine made by a method described herein, including in aspects, embodiments, examples, tables, figures, and claims. Provided herein is a reconstituted liquid vaccine comprising a dry vaccine as described herein (including in an aspect, embodiment, example, table, figure, or claim) or a dry vaccine prepared using a method as described herein (including in an aspect, embodiment, example, table, figure, or claim) and a solvent (e.g., water, buffer, solution, liquid including an excipient).

Provided in another aspect is a pharmaceutical composition including a pharmaceutically acceptable excipient and any of the compositions (e.g., vaccines) described herein (including embodiment).

The compositions described herein (including embodiments and examples) can be administered alone or can be coadministered to the patient. Coadministration is meant to include simultaneous or sequential administration of the compositions individually or in combination (more than one composition). Thus, the preparations can also be combined, when desired, with other active substances (e.g., to reduce metabolic degradation, increase immune response (e.g., adjuvants)). An example of coadministration of vaccine compositions is a prime-boost method of administration.

Pharmaceutical compositions provided by the present disclosure include compositions wherein the active ingredient (e.g., compositions described herein, including embodiments) is contained in a therapeutically or prophylactically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., prevent infection, and/or reducing, eliminating, or slowing the progression of disease symptoms. Determination of a therapeutically or prophylactically effective amount of a composition of the disclosure is well within the capabilities of those skilled in the art, especially in light of the detailed disclosure herein.

### III. Methods

In an aspect is provided a method for preparing a vaccine thin film including applying a liquid vaccine to a freezing surface; allowing the liquid vaccine to disperse and freeze on the freezing surface thereby forming a vaccine thin film.

In embodiments, the liquid vaccine includes an excipient. In embodiments, the liquid vaccine includes a plurality of different excipients. In embodiments, the excipient is a salt, buffer, detergent, polymer, amino acid, or preservative. In embodiments, the excipient is disodium edetate, sodium chloride, sodium citrate, sodium succinate, sodium hydroxide, sodium alginate, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, chitosan, Formaldehyde, Dulbecco's modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer. In embodiments, the excipient is trehalose. In embodiments, the liquid vaccine includes less than 5% wt/vol of the excipient/liquid vaccine. In embodiments, the liquid vaccine includes less than 4% wt/vol of the excipient/liquid vaccine. In embodiments, the liquid vaccine includes less than 3% wt/vol of the excipient/liquid vaccine. In embodiments, the liquid vaccine includes less than 2% wt/vol of the excipient/liquid vaccine. In embodiments, the liquid vaccine includes less than 1% wt/vol of the excipient/liquid vaccine. In embodiments, the liquid vaccine includes less than 0.5% wt/vol of the excipient/liquid vaccine. In embodiments, the liquid vaccine includes about 5% wt/vol of the excipient/liquid vaccine. In embodiments, the liquid vaccine includes about 4% wt/vol of the excipient/liquid vaccine. In embodiments, the liquid vaccine includes about 3% wt/vol of the excipient/liquid vaccine. In embodiments, the liquid vaccine includes about 2% wt/vol of the excipient/liquid vaccine. In embodiments, the liquid vaccine includes about 1% wt/vol of the excipient/liquid vaccine. In embodiments, the liquid vaccine includes about 0.5% wt/vol of the excipient/liquid vaccine. In embodiments, the liquid vaccine includes about 0.1, 0.2, 0.3, 0.4, 0.5, 0.6, 0.7, 0.8, 0.9, 1, 2, 3, 4, 5, 6, 7, 8, 9, or 10% (wt/vol) of the excipient/liquid vaccine. In embodiments, the liquid vaccine includes 0.1, 0.2, 0.3, 0.4, 0.5, 0.6, 0.7, 0.8, 0.9, 1, 2, 3, 4, 5, 6, 7, 8, 9, or 10% (wt/vol) of the excipient/liquid vaccine. In embodiments, the liquid vaccine includes less than 5% of the excipient. In embodiments, the liquid vaccine includes less than 4% of the excipient. In embodiments, the liquid vaccine includes less than 3% of the excipient. In embodiments, the liquid vaccine includes less than 2% of the excipient. In embodiments, the liquid vaccine includes less than 1% of the excipient. In embodiments, the liquid vaccine includes less than 0.5% of the excipient. In embodiments, the liquid vaccine includes about 5% of the excipient. In embodiments, the liquid vaccine includes about 4% of the excipient. In embodiments, the liquid vaccine includes about 3% of the excipient. In embodiments, the liquid vaccine includes about 2% of the excipient. In embodiments, the liquid vaccine includes about 1% of the excipient. In embodiments, the liquid vaccine includes about 0.5% of the excipient. In embodiments, the liquid vaccine includes about 0.1, 0.2, 0.3, 0.4, 0.5, 0.6, 0.7, 0.8, 0.9, 1, 2, 3, 4, 5, 6, 7, 8, 9, or 10% of the excipient. In embodiments, the liquid vaccine includes 0.1, 0.2, 0.3, 0.4, 0.5, 0.6, 0.7, 0.8, 0.9, 1, 2, 3, 4, 5, 6, 7, 8, 9, or 10% of the excipient.

In embodiments, the applying includes spraying or dripping droplets of the liquid vaccine. In embodiments, the vapor-liquid interface of the droplets is less than 500 cm⁻¹ area/volume. In embodiments, the vapor-liquid interface of the droplets is less than 400 cm⁻¹ area/volume. In embodiments, the vapor-liquid interface of the droplets is less than 300 cm⁻¹ area/volume. In embodiments, the vapor-liquid interface of the droplets is less than 200 cm⁻¹ area/volume. In embodiments, the vapor-liquid interface of the droplets is less than 100 cm⁻¹ area/volume. In embodiments, the vapor-liquid interface of the droplets is less than 50 cm⁻¹ area/volume. In embodiments, the vapor-liquid interface of the droplets is less than 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 cm⁻¹ area/volume.

In embodiments, the method further includes contacting the droplets with a freezing surface having a temperature below the freezing temperature of the liquid vaccine (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100° C. below the freezing temperature). In embodiments, the method further includes contacting the droplets with a freezing surface having a temperature differential of at least 30° C. between the droplets and the surface. In embodiments, the temperature differential is at least 40° C. between the droplets and the surface. In embodiments, the temperature differential is at least 50° C. between the droplets and the surface. In embodiments, the temperature differential is at least 60° C. between the droplets and the surface. In embodiments, the temperature differential is at least 70° C. between the droplets and the surface. In embodiments, the temperature differential is at least 80° C. between the droplets and the surface. In embodiments, the temperature differential is at least 90° C. between the droplets and the surface. In embodiments, the temperature differential between the droplets and the surface is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 180 or 200° C.

In embodiments, the vaccine thin film has a thickness of about 5 mm, about 4 mm, about 3 mm, about 2 mm, about 1 mm, less than 3 mm, less than 2 mm, less than Imm, less than 500 micrometers. In embodiments, the vaccine thin film has a thickness of less than 400 micrometers. In embodiments, the vaccine thin film has a thickness of less than 300 micrometers. In embodiments, the vaccine thin film has a thickness of less than 200 micrometers. In embodiments, the vaccine thin film has a thickness of less than 100 micrometers. In embodiments, the vaccine thin film has a thickness of less than 50 micrometers. In embodiments, the vaccine thin film has a thickness of less than 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 micrometers. In embodiments, the vaccine thin film has a thickness of about 500 micrometers. In embodiments, the vaccine thin film has a thickness of about 400 micrometers. In embodiments, the vaccine thin film has a thickness of about 300 micrometers. In embodiments, the vaccine thin film has a thickness of about 200 micrometers. In embodiments, the vaccine thin film has a thickness of about 100 micrometers. In embodiments, the vaccine thin film has a thickness of about 50 micrometers. In embodiments, the vaccine thin film has a thickness of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 micrometers.

In embodiments, the vaccine thin film has a surface area to volume ratio of between about 5 and 500 cm⁻¹. In embodiments, the vaccine thin film has a surface area to volume ratio of between 25 and 400 cm⁻¹. In embodiments, the vaccine thin film has a surface area to volume ratio of between 25 and 300 cm⁻¹. In embodiments, the vaccine thin film has a surface area to volume ratio of between 25 and 200 cm⁻¹. In embodiments, the vaccine thin film has a surface area to volume ratio of between 25 and 100 cm⁻¹. In embodiments, the vaccine thin film has a surface area to volume ratio of between 100 and 500 cm⁻¹. In embodiments, the vaccine thin film has a surface area to volume ratio of between 200 and 500 cm⁻¹. In embodiments, the vaccine thin film has a surface area to volume ratio of between 300 and 500 cm⁻¹. In embodiments, the vaccine thin film has a surface area to volume ratio of between 400 and 500 cm⁻¹. In embodiments, the vaccine thin film has a surface area to volume ratio of between 100 and 400 cm⁻¹. In embodiments, the vaccine thin film has a surface area to volume ratio of between 200 and 300 cm⁻¹. In embodiments, the vaccine thin film has a surface area to volume ratio of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 cm⁻¹. In embodiments, the vaccine thin film has a surface area to volume ratio of between about 25 and about 500 cm⁻¹. In embodiments, the vaccine thin film has a surface area to volume ratio of between about 25 and about 400 cm⁻¹. In embodiments, the vaccine thin film has a surface area to volume ratio of between about 25 and about 300 cm⁻¹. In embodiments, the vaccine thin film has a surface area to volume ratio of between about 25 and about 200 cm⁻¹. In embodiments, the vaccine thin film has a surface area to volume ratio of between about 25 and about 100 cm⁻¹. In embodiments, the vaccine thin film has a surface area to volume ratio of between about 100 and about 500 cm⁻¹. In embodiments, the vaccine thin film has a surface area to volume ratio of between about 200 and about 500 cm⁻¹. In embodiments, the vaccine thin film has a surface area to volume ratio of between about 300 and about 500 cm⁻¹. In embodiments, the vaccine thin film has a surface area to volume ratio of between about 400 and about 500 cm⁻¹. In embodiments, the vaccine thin film has a surface area to volume ratio of between about 100 and about 400 cm⁻¹. In embodiments, the vaccine thin film has a surface area to volume ratio of between about 200 and about 300 cm⁻¹.

In embodiments, the freezing rate of the droplets is between about 10 K/second and about 10⁵ K/second. In embodiments, the freezing rate of the droplets is between about 10 K/second and about 10⁴ K/second. In embodiments, the freezing rate of the droplets is between about 10 K/second and about 10³ K/second. In embodiments, the freezing rate of the droplets is between about 10² K/second and about 10³ K/second. In embodiments, the freezing rate of the droplets is between about 50 K/second and about 5×10² K/second. In embodiments, the freezing rate of the droplets is between 10 K/second and 10 K/second. In embodiments, the freezing rate of the droplets is between 10 K/second and 10⁴ K/second. In embodiments, the freezing rate of the droplets is between 10 K/second and 10³ K/second. In embodiments, the freezing rate of the droplets is between 10² K/second and 10³ K/second. In embodiments, the freezing rate of the droplets is between 50 K/second and 5×10² K/second. In embodiments, the freezing rate of the droplets is about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 K/second. In embodiments, the freezing rate of the droplets is 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 K/second. In embodiments, each of the droplets freezes upon contact with the freezing surface in less than about 50, 75, 100, 125, 150, 175, 200, 250, 500, 1,000, or 2,000 milliseconds. In embodiments, each of the droplets freezes upon contact with the freezing surface in less than 50, 75, 100, 125, 150, 175, 200, 250, 500, 1,000, or 2,000 milliseconds.

In embodiments, the droplets have an average diameter between about 0.1 and about 5 mm, between about 2 and about 25° C. In embodiments, the droplets have an average diameter between about 2 and about 4 mm, between about 20 and about 25° C. In embodiments, the droplets have an average diameter between about 1 and about 4 mm, between about 2 and about 25° C. In embodiments, the droplets have an average diameter between about 2 and about 3 mm, between about 2 and about 25° C. In embodiments, the droplets have an average diameter between about 1 and about 3 mm, between about 2 and about 25° C. In embodiments, the droplets have an average diameter between about 1 and about 2 mm, between about 2 and about 25° C. In embodiments, the droplets have an average diameter between about 3 and about 4 mm, between about 2 and about 25° C. In embodiments, the droplets have an average diameter between 0.1 and 5 mm, between 2 and 25° C. In embodiments, the droplets have an average diameter between 2 and 4 mm, between 2 and 25° C. In embodiments, the droplets have an average diameter between 1 and 4 mm, between 2 and 25° C. In embodiments, the droplets have an average diameter between 2 and 3 mm, between 2 and 25° C. In embodiments, the droplets have an average diameter between 1 and 3 mm, between 2 and 25° C. In embodiments, the droplets have an average diameter between 1 and 2 mm, between 2 and 25° C. In embodiments, the droplets have an average diameter between 3 and 4 mm, between 2° and 25° C.

In embodiments, the method further includes removing the solvent (e.g., water or liquid) from the vaccine thin film to form a dry vaccine.

In embodiments, is a method of making a dry vaccine from a vaccine thin film (e.g., including a vaccine thin film made using a method as described herein), including removing the solvent (e.g., water or liquid) from the vaccine thin film to form a dry vaccine. In embodiments of the methods described herein, the dry vaccine is a dry vaccine as described herein, including in an aspect, embodiment, example, table, figure, or claim. In embodiments, a method of making a vaccine thin film or a method of making dry vaccine is used to make a dry vaccine as described herein, including in an aspect, embodiment, example, table, figure, or claim.

In embodiments, the removing of the solvent includes lyophilization. In embodiments, the removing of the solvent includes lyophilization at temperatures of −20° C. or less. In embodiments, the removing of the solvent includes lyophilization at temperatures of −25° C. or less. In embodiments, the solvent includes lyophilization at temperatures of −40° C. or less. In embodiments, the removing of the solvent includes lyophilization at temperatures of −50° C. or less. In embodiments, the removing of the solvent includes lyophilization at temperatures of about −20° C. or less. In embodiments, the removing of the solvent includes lyophilization at temperatures of about −25° C. or less. In embodiments, the solvent includes lyophilization at temperatures of about −40° C. or less. In embodiments, the removing of the solvent includes lyophilization at temperatures of about −50° C. or less. Primary drying can be performed at −20° C. to −50° C., and secondary drying can be performed at 4-25° C.

In embodiments, the method further includes solvating, reconstituting or rehydrating the dry vaccine thereby forming a reconstituted liquid vaccine. A reconstituted liquid vaccine may also be called a solvated dry vaccine.

In embodiments, is a method of making a reconstituted liquid vaccine from a dry vaccine (e.g., including a dry vaccine made using a method as described herein), including solvating a dry vaccine and thereby forming a reconstituted liquid vaccine. In embodiments of the methods described herein, the dry vaccine is a dry vaccine as described herein, including in an aspect, embodiment, example, table, figure, or claim. In embodiments, a method of making a vaccine thin film, a method of making a dry vaccine, or a method of reconstituting a liquid vaccine is used to make a reconstituted liquid vaccine as described herein, including in an aspect, embodiment, example, table, figure, or claim.

In embodiments, the immunogenicity of the reconstituted liquid vaccine is at least 60% the immunogenicity of the liquid vaccine (prior to forming the dry vaccine from the liquid vaccine). In embodiments, the immunogenicity of the reconstituted liquid vaccine is at least 70% the immunogenicity of the liquid vaccine (prior to forming the dry vaccine from the liquid vaccine). In embodiments, the immunogenicity of the reconstituted liquid vaccine is at least 80% the immunogenicity of the liquid vaccine (prior to forming the dry vaccine from the liquid vaccine). In embodiments, the immunogenicity of the reconstituted liquid vaccine is at least 90% the immunogenicity of the liquid vaccine (prior to forming the dry vaccine from the liquid vaccine). In embodiments, the immunogenicity of the reconstituted liquid vaccine is at least 95% the immunogenicity of the liquid vaccine (prior to forming the dry vaccine from the liquid vaccine). In embodiments, the immunogenicity of the reconstituted liquid vaccine is at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% the immunogenicity of the liquid vaccine (prior to forming the dry vaccine from the liquid vaccine). In embodiments, the immunogenicity of the reconstituted liquid vaccine is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% the immunogenicity of the liquid vaccine (prior to forming the dry vaccine from the liquid vaccine).

In embodiments, the reconstituted liquid vaccine includes particles. In embodiments, the particles have an average diameter of between about 10 nm and about 2 μm. In embodiments, the particles have an average diameter of between about 20 nm and about 2 μm. In embodiments, the particles have an average diameter of between about 50 nm and about 2 μm. In embodiments, the particles have an average diameter of between about 100 nm and about 2 μm. In embodiments, the particles have an average diameter of between about 200 nm and about 2 μm. In embodiments, the particles have an average diameter of between about 500 nm and about 2 μm. In embodiments, the particles have an average diameter of between about 1 μm and about 2 μm. In embodiments, the particles have an average diameter of between about 10 nm and about 1 μm. In embodiments, the particles have an average diameter of between about 10 nm and about 500 nm. In embodiments, the particles have an average diameter of between about 10 nm and about 200 nm. In embodiments, the particles have an average diameter of between about 10 nm and about 200 nm. In embodiments, the particles have an average diameter of between about 10 nm and about 100 nm. In embodiments, the particles have an average diameter of between about 10 nm and about 50 nm. In embodiments, the particles have an average diameter of between about 10 nm and about 20 nm. In embodiments, the particles have an average diameter of between about 20 nm and about 1 μm. In embodiments, the particles have an average diameter of between about 50 nm and about 500 nm. In embodiments, the particles have an average diameter of between about 100 nm and about 500 nm. In embodiments, the particles have an average diameter of between about 100 nm and about 200 nm. In embodiments, the reconstituted liquid vaccine includes particles. In embodiments, the particles have an average diameter of between 10 nm and 2 μm. In embodiments, the particles have an average diameter of between 20 nm and 2 μm. In embodiments, the particles have an average diameter of between 50 nm and 2 μm. In embodiments, the particles have an average diameter of between 100 nm and 2 μm. In embodiments, the particles have an average diameter of between 200 nm and 2 μm. In embodiments, the particles have an average diameter of between 500 nm and 2 μm. In embodiments, the particles have an average diameter of between 1 μm and 2 μm. In embodiments, the particles have an average diameter of between 10 nm and 1 μm. In embodiments, the particles have an average diameter of between 10 nm and 500 nm. In embodiments, the particles have an average diameter of between 10 nm and 200 nm. In embodiments, the particles have an average diameter of between 10 nm and 200 nm. In embodiments, the particles have an average diameter of between 10 nm and 100 nm. In embodiments, the particles have an average diameter of between 10 nm and 50 nm. In embodiments, the particles have an average diameter of between 10 nm and 20 nm. In embodiments, the particles have an average diameter of between 20 nm and 1 μm. In embodiments, the particles have an average diameter of between 50 nm and 500 nm. In embodiments, the particles have an average diameter of between 100 nm and 500 nm. In embodiments, the particles have an average diameter of between 100 nm and 200 nm.

In embodiments, the reconstituted liquid vaccine includes particles of the same average diameter as the liquid vaccine (prior to forming the dry vaccine from the liquid vaccine) particles. In embodiments, the reconstituted liquid vaccine includes particles having an average diameter within 5% of the average diameter of particles in the liquid vaccine (prior to forming the dry vaccine from the liquid vaccine). In embodiments, the reconstituted liquid vaccine includes particles having an average diameter within 10% of the average diameter of particles in the liquid vaccine (prior to forming the dry vaccine from the liquid vaccine). In embodiments, the reconstituted liquid vaccine includes particles having an average diameter within 20% of the average diameter of particles in the liquid vaccine (prior to forming the dry vaccine from the liquid vaccine). In embodiments, the reconstituted liquid vaccine includes particles having an average diameter within 10%, 20%, 30% or 40% of the average diameter of particles in the liquid vaccine (prior to forming the dry vaccine from the liquid vaccine). There may less than 5% or 10% of the particles aggregated (e.g., 8 particles can aggregate together to form a single particle with a diameter approximately that of the original smaller particles). In embodiments, the reconstituted liquid vaccine includes particles having an average diameter within about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30% of the average diameter of particles in the liquid vaccine (prior to forming the dry vaccine from the liquid vaccine). In embodiments, the reconstituted liquid vaccine includes particles having an average diameter within 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30% of the average diameter of particles in the liquid vaccine (prior to forming the dry vaccine from the liquid vaccine).

In embodiments, the solvating, reconstituting or rehydrating of the dry vaccine is at least one day after preparing the dry vaccine from the liquid vaccine (e.g., the dry vaccine is stored for at least one day). In embodiments, the solvating of the dry vaccine is at least two days after preparing the dry vaccine from the liquid vaccine (e.g., the dry vaccine is stored for at least two days). In embodiments, the solvating of the dry vaccine is at least three days after preparing the dry vaccine from the liquid vaccine (e.g., the dry vaccine is stored for at least three days). In embodiments, the solvating of the dry vaccine is at least one week after preparing the dry vaccine from the liquid vaccine (e.g., the dry vaccine is stored for at least one week). In embodiments, the solvating of the dry vaccine is at least two weeks after preparing the dry vaccine from the liquid vaccine (e.g., the dry vaccine is stored for at least two weeks). In embodiments, the solvating of the dry vaccine is at least one month after preparing the dry vaccine from the liquid vaccine (e.g., the dry vaccine is stored for at least one month). In embodiments, the solvating of the dry vaccine is at least two months after preparing the dry vaccine from the liquid vaccine (e.g., the dry vaccine is stored for at least two months). In embodiments, the solvating of the dry vaccine is at least three months after preparing the dry vaccine from the liquid vaccine (e.g., the dry vaccine is stored for at least three months). In embodiments, the solvating of the dry vaccine is at least six months after preparing the dry vaccine from the liquid vaccine (e.g., the dry vaccine is stored for at least six months). In embodiments, the solvating of the dry vaccine is at least one year after preparing the dry vaccine from the liquid vaccine (e.g., the dry vaccine is stored for at least one year). In embodiments, the solvating of the dry vaccine is at least two years after preparing the dry vaccine from the liquid vaccine (e.g., the dry vaccine is stored for at least two years). In embodiments, the solvating of the dry vaccine is at least three years after preparing the dry vaccine from the liquid vaccine (e.g., the dry vaccine is stored for at least three years). In embodiments, the solvating of the dry vaccine is at least five years after preparing the dry vaccine from the liquid vaccine (e.g., the dry vaccine is stored for at least five years). In embodiments, the solvating of the dry vaccine is at least ten years after preparing the dry vaccine from the liquid vaccine (e.g., the dry vaccine is stored for at least ten years).

In embodiments, prior to the solvating, reconstituting or rehydrating of the dry vaccine, the dry vaccine is stored at about 4° C. for at least 99% of the time. In embodiments, prior to the solvating of the dry vaccine, the dry vaccine is stored at less than 4° C. for at least 99% of the time. In embodiments, prior to the solvating of the dry vaccine, the dry vaccine is stored at less than 0° C. for at least 99% of the time. In embodiments, prior to the solvating of the dry vaccine, the dry vaccine is stored at less than −20° C. for at least 99% of the time. In embodiments, prior to the solvating of the dry vaccine, the dry vaccine is stored at about −20° C. for at least 99% of the time. In embodiments, prior to the solvating of the dry vaccine, the dry vaccine is stored at less than −80° C. for at least 99% of the time. In embodiments, prior to the solvating of the dry vaccine, the dry vaccine is stored at about −80° C. for at least 99% of the time. In embodiments, prior to the solvating of the dry vaccine, the dry vaccine is stored at ambient temperatures (e.g., room temperature). In embodiments, prior to the solvating of the dry vaccine, the dry vaccine is stored at between 20 and 24° C. for at least 99% of the time. In embodiments, prior to the solvating of the dry vaccine, the dry vaccine is stored at between 4 and 24° C. for at least 99% of the time. In embodiments, prior to the solvating of the dry vaccine, the dry vaccine is stored at between 0 and 24° C. for at least 99% of the time. In embodiments, prior to the solvating of the dry vaccine, the dry vaccine is stored at between 4 and 40° C. for at least 99% of the time. In embodiments, prior to the solvating of the dry vaccine, the dry vaccine is stored at between 0 and 40° C. for at least 99% of the time. In embodiments, prior to the solvating of the dry vaccine, the dry vaccine is stored at about 4° C. for at least 90% of the time. In embodiments, prior to the solvating of the dry vaccine, the dry vaccine is stored at less than 4° C. for at least 90% of the time. In embodiments, prior to the solvating of the dry vaccine, the dry vaccine is stored at less than 0° C. for at least 90% of the time. In embodiments, prior to the solvating of the dry vaccine, the dry vaccine is stored at less than −20° C. for at least 90% of the time. In embodiments, prior to the solvating of the dry vaccine, the dry vaccine is stored at between 20 and 24° C. for at least 90% of the time. In embodiments, prior to the solvating of the dry vaccine, the dry vaccine is stored at between 4 and 24° C. for at least 90% of the time. In embodiments, prior to the solvating of the dry vaccine, the dry vaccine is stored at between 0 and 24° C. for at least 90% of the time. In embodiments, prior to the solvating of the dry vaccine, the dry vaccine is stored at between 4 and 40° C. for at least 90% of the time. In embodiments, prior to the solvating of the dry vaccine, the dry vaccine is stored at between 0 and 40° C. for at least 90% of the time.

In embodiments, upon solvating, reconstituting or rehydrating the dry vaccine the resulting reconstituted liquid vaccine remains homogeneous. As used in reference to the status of a reconstituted liquid vaccine, the term “homogenous” refers to a lack of a significant amount of aggregation and/or precipitation forming, such that the reconstituted liquid vaccine does not include solid matter that is not evenly dispersed (e.g., solid matter visible to the naked eye, solid matter that settles in the liquid, solid matter that was not apparent in a liquid vaccine prior to formation of the dry vaccine and reconstitution, precipitate that was not present in the liquid vaccine prior to formation of the dry vaccine). A homogenous reconstituted liquid sample may include particles of antigenic protein adsorbed to the adjuvant (e.g., that are suspended or dispersed in the reconstituted liquid vaccine). In embodiments, upon solvating the dry vaccine the resulting reconstituted liquid vaccine remains homogeneous for at least one day. In embodiments, upon solvating the dry vaccine the resulting reconstituted liquid vaccine remains homogeneous for at least two days. In embodiments, upon solvating the dry vaccine the resulting reconstituted liquid vaccine remains homogeneous for at least three days. In embodiments, upon solvating the dry vaccine the resulting reconstituted liquid vaccine remains homogeneous for at least one week. In embodiments, upon solvating the dry vaccine the resulting reconstituted liquid vaccine remains homogeneous for at least two weeks. In embodiments, upon solvating the dry vaccine the resulting reconstituted liquid vaccine remains homogeneous for at least one month. In embodiments, upon solvating the dry vaccine the resulting reconstituted liquid vaccine remains homogeneous for at least three months. In embodiments, upon solvating the dry vaccine the resulting reconstituted liquid vaccine remains homogeneous for at least six months. In embodiments, upon solvating the dry vaccine the resulting reconstituted liquid vaccine remains homogeneous for at least one year. In embodiments, upon solvating the dry vaccine the resulting reconstituted liquid vaccine does not form a precipitate (e.g., solid matter visible to the naked eye, solid matter that settles in the liquid, solid matter that was not apparent in a liquid vaccine prior to formation of the dry vaccine and reconstitution, precipitate that was not present in the liquid vaccine prior to formation of the dry vaccine). In embodiments, upon solvating the dry vaccine the resulting reconstituted liquid vaccine does not form a precipitate for at least one day. In embodiments, upon solvating the dry vaccine the resulting reconstituted liquid vaccine does not form a precipitate for at least two days. In embodiments, upon solvating the dry vaccine the resulting reconstituted liquid vaccine does not form a precipitate for at least three days. In embodiments, upon solvating the dry vaccine the resulting reconstituted liquid vaccine does not form a precipitate for at least one week. In embodiments, upon solvating the dry vaccine the resulting reconstituted liquid vaccine does not form a precipitate for at least two weeks. In embodiments, upon solvating the dry vaccine the resulting reconstituted liquid vaccine does not form a precipitate for at least one month. In embodiments, upon solvating the dry vaccine the resulting reconstituted liquid vaccine does not form a precipitate for at least three months. In embodiments, upon solvating the dry vaccine the resulting reconstituted liquid vaccine does not form a precipitate for at least six months. In embodiments, upon solvating the dry vaccine the resulting reconstituted liquid vaccine does not form a precipitate for at least one year.

In embodiments, the precipitate includes particles having an average diameter greater than 50 μm. In embodiments, the precipitate includes particles having an average diameter greater than 100 μm. In embodiments, the precipitate includes particles having an average diameter greater than 200 μm. In embodiments, the precipitate includes particles having an average diameter greater than 300 μm. In embodiments, the precipitate includes particles having an average diameter greater than 400 μm. In embodiments, the precipitate includes particles having an average diameter greater than 500 μm. In embodiments, the precipitate includes particles having an average diameter greater than 600 μm. In embodiments, the precipitate includes particles having an average diameter greater than 700 μm. In embodiments, the precipitate includes particles having an average diameter greater than 800 μm. In embodiments, the precipitate includes particles having an average diameter greater than 900 μm. In embodiments, the precipitate includes particles having an average diameter greater than 1000 μm. In embodiments, the precipitate includes particles having an average diameter greater than about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μm. In embodiments, the precipitate includes particles having an average diameter of about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μm. In embodiments, the precipitate includes particles having an average diameter greater than 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μm. In embodiments, the precipitate (that is not formed) includes particles having an average diameter of 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μm. In embodiments, the precipitate (that is not formed) includes at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of the total antigenic protein in the reconstituted liquid vaccine. In embodiments, upon solvating the dry vaccine the resulting reconstituted liquid vaccine does not form a precipitate including more than about 1% of the total antigenic protein in the reconstituted liquid vaccine. In embodiments, upon solvating the dry vaccine the resulting reconstituted liquid vaccine does not form a precipitate including more than about 2% of the total antigenic protein in the reconstituted liquid vaccine. In embodiments, upon solvating the dry vaccine the resulting reconstituted liquid vaccine does not form a precipitate including more than about 3% of the total antigenic protein in the reconstituted liquid vaccine. In embodiments, upon solvating the dry vaccine the resulting reconstituted liquid vaccine does not form a precipitate including more than about 4% of the total antigenic protein in the reconstituted liquid vaccine. In embodiments, upon solvating the dry vaccine the resulting reconstituted liquid vaccine does not form a precipitate including more than about 5% of the total antigenic protein in the reconstituted liquid vaccine. In embodiments, upon solvating the dry vaccine the resulting reconstituted liquid vaccine does not form a precipitate including more than about 1, 2, 3, 4, 5, 6, 7, 8, 9, 10, 11, 12, 13, 14, 15, 16, 17, 18, 19, or 20% of the total antigenic protein in the reconstituted liquid vaccine. In embodiments the precipitate includes irreversible aggregates of antigenic protein and/or adjuvant.

In embodiments, the liquid vaccine includes a commercially available vaccine. In embodiments, the liquid vaccine is a commercially available vaccine. In embodiments, the liquid vaccine has received market approval from the US FDA or the corresponding authority in another country. In embodiments, the liquid vaccine is a vaccine for the treatment of diphtheria, tetanus, pertussis, influenza, pneumonia, otitis media, bacteremia, meningitis, hepatitis, cirrhosis, anthrax poisoning, botulism, rabies, warts, poliomyelitis, Japanese encephalitis, Coronavirus disease, or cancer. In embodiments, the liquid vaccine is a vaccine for the treatment of infection by Clostridium tetani, Clostridium botulinum, Streptococcus pneumonia, Hepatitis A, Hepatitis B, Haemophilus influenza, Corynebacterium diphtheria, Bordetella pertussis, Human papillomavirus, Bacillus anthracis, Rabies virus, Japanese encephalitis virus, SARS-associated Coronavirus, or Poliovirus. In embodiments, the liquid vaccine includes a commercially available vaccine and another component not included in the commercially available vaccine (e.g., an excipient (e.g., trehalose)).

In an aspect is provided a method of treating a disease in a patient in need of such treatment, the method including administering a therapeutically effective amount of a solvated dry vaccine as described herein (e.g., in an aspect, embodiment, example, table, figure, or claims) (e.g., a reconstituted liquid vaccine as described herein) to the patient.

In embodiments, the disease is diphtheria, tetanus, pertussis, influenza, pneumonia, otitis media, bacteremia, meningitis, hepatitis, cirrhosis, anthrax poisoning, rabies, warts, poliomyelitis, Japanese encephalitis, Coronavirus disease, or cancer. In embodiments, the disease is caused by an infectious agent. In embodiments, the infectious agent is a bacterium. In embodiments, the infectious agent is a virus. In embodiments, the infectious agent is Clostridium tetani, Clostridium botulinum, Streptococcus pneumonia, Hepatitis A, Hepatitis B, Haemophilus influenza, Corynebacterium diphtheria, Bordetella pertussis, Human papillomavirus, Bacillus anthracis, Rabies virus, Japanese encephalitis virus, SARS-associated coronavirus, or Poliovirus.

In an aspect is provided a method of treating a disease in a patient in need of such treatment, the method including administering a therapeutically effective amount of dry vaccine as described herein (e.g., in an aspect, embodiment, example, table, figure, or claims) (e.g., a reconstituted liquid vaccine as described herein) to the patient.

In embodiments, the disease is diphtheria, tetanus, pertussis, influenza, pneumonia, otitis media, bacteremia, meningitis, hepatitis, cirrhosis, anthrax poisoning, botulism, rabies, warts, poliomyelitis, Japanese encephalitis, Coronavirus disease, or cancer. In embodiments, the disease is caused by an infectious agent. In embodiments, the infectious agent is a bacterium. In embodiments, the infectious agent is a virus. In embodiments, the infectious agent is Clostridium tetani, Clostridium botulinum, Streptococcus pneumonia, Hepatitis A, Hepatitis B, Haemophilus influenza, Corynebacterium diphtheria, Bordetella pertussis, Human papillomavirus, Bacillus anthracis, Rabies virus, Japanese encephalitis virus, SARS-associated Coronavirus, or Poliovirus.

In embodiments, the dry vaccine is administered by inhalation, intradermally, orally, or vaginally. In embodiments, the dry vaccine is administered through the nasal mucosa, bronchoalveolar mucosa, or gastrointestinal mucosa.

In embodiments, the method is a method described herein, including in an aspect, embodiment, example, table, figure, or claim. Provided herein is a method of preparing a dry vaccine including a method of preparing a vaccine thin film as described herein (including in an aspect, embodiment, example, table, figure, or claim) and a method of removing a solvent from a vaccine thin film as described herein (including in an aspect, embodiment, example, table, figure, or claim). Provided herein is a method of preparing a reconstituted dry vaccine including a method of preparing a dry vaccine as described herein (including in an aspect, embodiment, example, table, figure, or claim), a method of preparing a vaccine thin film as described herein (including in an aspect, embodiment, example, table, figure, or claim) and a method of removing a solvent from a vaccine thin film as described herein (including in an aspect, embodiment, example, table, figure, or claim).

In embodiments, to form a powder vaccine, an aqueous vaccine composition is first frozen to form a frozen vaccine composition, then the frozen water is removed to form the vaccine powder. A fast-freezing process is used to form the frozen vaccine composition. A fast-freezing process, as used herein, is a process that can freeze a thin film of liquid (less than about 500 microns or 2-4 mm) in a time of less than or equal to about 3000 milliseconds. In the TFF process liquid droplets fall from a given height and impact, spread, and freeze on a cooled solid substrate. Typically, the substrate is a metal drum that is cooled to below 250° K, or below 200° K or below 150° K. On impact the droplets that are deformed into thin films freeze in a time of between about 70 ms and 3000 ms. The frozen thin films may be removed from the substrate by a stainless-steel blade mounted along the rotating drum surface. The frozen thin films are collected in liquid nitrogen to maintain in the frozen state. Further details regarding thin film freezing processes may be found in the paper to Engstrom et al. “Formation of Stable Submicron Protein Particles by Thin Film Freezing” Pharmaceutical Research, Vol. 25, No. 6, June 2008, 1334-1346, which is incorporated herein by reference.

Water (e.g., frozen water) is removed from the frozen vaccine composition to produce a vaccine powder. Water (e.g., frozen water) may be removed by a lyophilization process or a freeze-drying process. Water may also be removed by an atmospheric freeze-drying process.

The resulting vaccine powder can be readily reconstituted to form a stable dispersion without significant loss of stability or activity. The vaccine powder may be transported and stored in a wide range of temperatures without concern of accidental exposure to freezing conditions. In addition, the vaccine powder may also be stored at room temperature, which will potentially decrease the costs of vaccines. In fact, it is generally less costly to transport dry solid powder than liquid.

Currently human vaccines (e.g., marketed and/or approved human vaccines, such as FDA approved human vaccines) that have an adjuvant are all administered by needle-syringe-based injections. It would be beneficial to patients and the healthcare system if the vaccines were administered non-invasively without hypodermic needles. The dried vaccine powder can potentially be administered by an alternative route such as, but not limited to, inhalation as a dried powder, transcutaneously using a patch with or without microneedles, intradermally using a solid jet injection device (e.g., powder jet injector), orally in tablets or capsules, buccally in buccal tablets or films, or vaginally using a special vaginal drug delivery device. The above-mentioned routes of administration are not only more convenient and friendly to patients, but more importantly they can enable the induction of mucosal immune responses. Functional antibodies in the mucosal secretion (e.g., nasal mucus, bronchoalveolar mucus, or the gastrointestinal mucus) of a host can effectively neutralize pathogens or toxins even before they enter the host.

Described herein are compositions and methods for preparing a vaccine thin film or a dry vaccine by spraying or dripping droplets of a liquid vaccine such that the vaccine is exposed to an vapor-liquid interface of less than 500 cm⁻¹ area/volume, such as 25 to 500 cm⁻¹ (e.g., less than 50, 100, 150, 200, 250, 300, 400) and contacting the droplet with a freezing surface having a temperature lower than the freezing temperature of the liquid vaccine (e.g., has a temperature differential of at least 30° C. between the droplet and the surface), wherein the surface freezes the droplet into a thin film with a thickness of less than 5 mm, such as about 2-4 mm, about 1 mm, about 500 micrometers (e.g., 450, 400, 350, 300, 250, 200, 150, 100, or 50 micrometers). In embodiments, the method may further include the step of removing the liquid (e.g., solvent, water) from the frozen material to form a dry vaccine (e.g., particles). In embodiments, the droplets freeze upon contact with the surface in less than 50, 75, 100, 125, 150, 175, 200, 250, 500, 1,000, 2,000, or 3000 milliseconds. In embodiments, the droplets freeze upon contact with the surface in less than 50 or 150 milliseconds. In embodiments, the droplet has a diameter between 2 and 5 mm at room temperature. In embodiments, the droplet forms a thin film on the freezing surface of between 50 micrometers and 5 mm, such as 2-4 mm in thickness. In embodiments, the droplets have a cooling rate of between 50-250 K/s. In embodiments, the particles of the dry vaccine, after liquid (e.g., solvent or water) removal, have a surface area of at least 10, 15, 25, 50, 75, 100, 125, 150 or 200 m²/gr (e.g., surface area of 10, 15, 25, 50, 75, 100, 125, 150 or 200 m²/gr). Minimizing gas-liquid interface can improve protein stability by limiting the amount of protein that can adsorb to the interface.

In embodiments, the droplets may be delivered to the cold or freezing surface in a variety of manners and configurations. In embodiments, the droplets may be delivered in parallel, in series, at the center, middle or periphery or a platen, platter, plate, roller, conveyor surface. In embodiments, the freezing or cold surface may be a roller, a belt, a solid surface, circular, cylindrical, conical, oval and the like that permit for the droplet to freeze. For a continuous process a belt, platen, plate or roller may be particularly useful. In embodiments, the frozen droplets may form beads, strings, films or lines of frozen liquid vaccine. In embodiments, the effective ingredient is removed from the surface with a scraper, wire, ultrasound or other mechanical separator prior to the lyophilization process. Once the material is removed from the surface of the belt, platen, roller or plate the surface is free to receive additional material.

In embodiments, the surface is cooled by a cryogenic solid, a cryogenic gas, a cryogenic liquid or a heat transfer fluid capable of reaching cryogenic temperatures or temperatures below the freezing point of the liquid vaccine (e.g., at least 30° C. less than the temperature of the droplet). In embodiments, the liquid vaccine further includes one or more excipients selected from surfactants, polymeric surfactants, vesicles, polymers, including copolymers and homopolymers and biopolymers, dispersion aids, and serum albumin. In embodiments, aggregation of the antigenic protein is less than 3% of the total antigenic protein in the vaccine (e.g., irreversible aggregation). In embodiments, the temperature differential between the droplet and the surface is at least 30° C. In embodiments, the excipients or stabilizers that can be included in the liquid vaccines that are to be frozen as described herein include: cryoprotectants, lyoprotectants, surfactants, fillers, stabilizers, polymers, protease inhibitors, antioxidants and absorption enhancers. Specific nonlimiting examples of excipients that may be included in the vaccines described herein include: Span 80, Tween 80, Brij 35, Brij 98, Pluronic, sucroester 7, sucroester 11, sucroester 15, sodium lauryl sulfate, oleic acid, laureth-9, laureth-8, lauric acid, vitamin E TPGS, Gelucire 50/13, Gelucire 53/10, Labrafil, glycolic acid and salts, deoxycholic acid and salts, sodium fusidate, cyclodextrins, polyethylene glycols, labrasol, polyvinyl alcohols, polyvinyl pyrrolidones and tyloxapol.

In embodiments, the method may further include the step of removing the liquid (e.g., solvent or water) from the frozen liquid vaccine to form a dry vaccine. In embodiments, the solvent further includes at least one or more excipient or stabilizers selected from, e.g., surfactants, polymeric surfactants, vesicles, polymers, including copolymers and homopolymers and biopolymers, dispersion aids, and serum albumin. In embodiments, the temperature differential between the solvent and the surface is at least about 30° C.

In embodiments, the resulting powder can be redispersed into a suitable aqueous medium such as saline, buffered saline, water, buffered aqueous media, solutions of amino acids, solutions of vitamins, solutions of carbohydrates, or the like, as well as combinations of any two or more thereof, to obtain a suspension that can be administered to mammals (e.g., humans).

In embodiments, is described a single-step, single-vial method for preparing a vaccine thin film or dry vaccine by reducing the temperature of a vial wherein the vial has a temperature below the freezing temperature of a liquid vaccine (e.g., a temperature differential of at least 30° C. between the liquid vaccine and the vial) and spraying or dripping droplets of a liquid vaccine directly into the vial such that the antigenic protein of the liquid vaccine is exposed to a vapor-liquid interface of less than 500 cm⁻¹ area/volume, wherein the surface freezes the droplet into a thin film with a thickness of less than 500 micrometers and a surface area to volume between 25 to 500 cm⁻¹. In embodiments, the droplets freeze upon contact with the surface in less than about 50, 75, 100, 125, 150, 175, 200, 250, 500, 1,000, 2,000 or 3000 milliseconds (e.g., in about 50, 75, 100, 125, 150, 175, 200, 250, 500, 1,000 or 2,000 or 3000 milliseconds), and may freeze upon contact with the surface in about 50 or 150 to 500 milliseconds. In embodiments, a droplet has a diameter between 0.1 and 5 mm at room temperature (e.g., a diameter between 2 and 4 mm at room temperature). In embodiments, the droplet forms a thin film on the surface of between 50 micrometers to 5 mm, such as about 2-4 millimeters in thickness. In embodiments, the droplets have a cooling rate of between 50-250 K/s. In embodiments, the vial may be cooled by a cryogenic solid, a cryogenic gas, a cryogenic liquid, a freezing fluid, a freezing gas, a freezing solid, a heat exchanger, or a heat transfer fluid capable of reaching cryogenic temperatures or temperatures below the freezing point of the liquid vaccine. In embodiments, the vial may be rotated as the spraying or droplets are delivered to permit the layering or one or more layers of the liquid vaccine. In embodiments, the vial and the liquid vaccine are pre-sterilized prior to spraying or dripping. In embodiments, the step of spraying or dripping is repeated to overlay one or more thin films on top of each other to fill the vial to any desired level up to totally full.

### IV. Examples

The following examples as well as the figures are included to demonstrate preferred embodiments of the disclosure. It should be appreciated by those of skill in the art that the techniques disclosed in the examples or figures represent techniques discovered by the inventors to function well in the practice of the disclosure and thus can be considered to constitute preferred modes for its practice. However, those of skill in the art should, in light of the disclosure, appreciate that many changes can be made in the specific embodiments which are disclosed and still obtain a like or similar result without departing from the spirit and scope of the disclosure.

### Example 1—Dry Powder Compositions of AS01_{B} Liposome-Based Adjuvant

Background. Adjuvants are employed to enhance a vaccine's immunogenicity (Di Pasquale et al., 2015). Adjuvant system01_{B} (i.e., AS01_{B}) is among a series of adjuvant systems developed by GlaxoSmithKline Vaccines (Wavre, Belgium) to elicit durable humoral and cellular immune responses (VandepapeliUre et al., 2008). AS01_{B} is a liposome formulation comprising two immunostimulants that work synergistically, namely 3-O-desacyl-4′-monophosphoryl lipid A (MPL) and QS-21, a highly purified triterpene glucoside (Didierlaurent et al., 2017).

AS01_{B} is currently utilized as an adjuvant in the FDA approved SHINGRIX® vaccine for human use (i.e., Zoster Vaccine Recombinant, Adjuvanted, suspension for intramuscular injection). In this case AS01_{B} is provided as a suspension in a vial, which is used to reconstitute lyophilized varicella zoster virus (VZV) glycoprotein E (gE) antigen in a separate vial immediately before administration. The AS01_{B} suspension and lyophilized gE antigen should be stored refrigerated between 2° C. and 8° C. and should not be frozen. The vaccine should be discarded if the adjuvant suspension and/or the lyophilized antigen have been accidently frozen. Herein, the inventors report a method to convert AS01_{B}-adjuvant to dry powder while maintaining its particle size distribution after reconstitution. This method has also maintained the particle size distribution of the AS01_{B} adjuvant after it was exposed to repeated freezing and thawing.

Materials and Methods. AS01_{B} was prepared in house as previously described with some modifications (Vandepapelire et al., 2008). Briefly, the liposome formulation was prepared by dissolving 1 mg of 1,2-dioleoyl-sn-glycero-3-phosphocholine (DOPC, Avanti, Alabaster, Ala., US), 0.25 mg of cholesterol (Avanti, Alabaster, Ala., US), and 50 μg MPL (Sigma Aldrich, St. Louis, Mo., US) in 2 mL ethanol. A lipid film was formed by solvent evaporation under a gentle stream of nitrogen gas. The lipid film was then hydrated with phosphate buffered saline (PBS, 5 mM, pH 7.4) to form liposomes. QS-21 (50 μg) in aqueous solution was added to the liposomes and the volume was adjusted to 0.5 mL using PBS.

Liquid AS01_{B} adjuvant was converted to dry powder using TFFD. Sucrose was employed as a cryoprotectant at the same concentration level (i.e., 4% w/v) employed in the lyophilized VZV glycoprotein E (gE) antigen of SHINGRIX® vaccine. Briefly, liquid AS01_{B} adjuvant was dropped onto a cryogenically cooled surface to form frozen thin films within rapidly. The frozen films were then lyophilized using a VirTis Advantage bench top tray lyophilizer (The VifTis Company, Inc. Gardiner, N.Y.). Lyophilization was performed over 60 h at pressures ≤100 mTorr. The shelf temperature was maintained at −40° C. for 20 h then gradually ramped to +25° C., over 20 h. Throughout the secondary drying phase, the vials were kept at +25° C. for additional 20 h. The vials were then stored at room temperature for future use.

To investigate the robustness of AS01_{B} powder to repeated freezing and thawing, liquid AS01_{B} adjuvant and its dry powder prepared by TFFD were exposed to three consecutive cycles of freezing at −20° C. for 8 h and thawing at 4° C. for 16 h. At the end of the third cycle, the powder was reconstituted with milli-Q water and its mean particle size was determined.

Dynamic light scattering (DLS) using a Malvern Zeta Sizer Nano ZS (MA, US) was employed to determine the particle size distribution and zeta potential of liquid AS01_{B} before (i.e., before TFFD) and rehydrated dry powder prepared by TFFD.

Results. The hypothesis that the ultra-rapid freezing rate of TFFD process does not adversely affect the particle size distribution of liposomes and thus liposome-based vaccine adjuvants was investigated using AS01_{B} adjuvant. As shown in FIG. 1A, the particle size distribution of liquid AS01_{B} adjuvant was maintained after TFFD when sucrose was employed as an excipient at a concentration of 77.39% w/w (i.e., 4% w/v). On the other hand, the zeta potential values of the AS01_{B} adjuvant significantly decreased after it was subjected to TFFD (p<0.01, FIG. 1B). As shown in FIG. 1C, the mean particle size of the AS01_{B} adjuvant did not significantly change after the dry powder was subjected to repeated freeze-and-thaw. However, subjecting the liquid adjuvant to repeated freeze-and-thaw caused significant aggregation (p<0.001).

### Example 2—Dry Powder Compositions of AS01_{B}-Adjuvanted OVA Model Vaccine

Materials and Methods. To prepare the model vaccine, ovalbumin (OVA, Sigma Aldrich, St. Louis, Mo., US) solution in PBS was added at a concentration of 50 μg/0.5 mL of the in house prepared AS01_{B} adjuvant. The applicability of TFFD process in converting a model vaccine to dry powder while maintaining its particle size distribution after reconstitution was investigated. For this purpose, sucrose was employed as a cryoprotectant at a concentration of 77.39% w/w (i.e., 4% w/v). Then, different sugars as well as a sugar alcohol were screened as possible cryoprotectants at different concentration levels. Various formulations (Table 1) containing different excipients (i.e., sucrose, trehalose or mannitol) at different concentration levels (i.e., 46.05, 63.1, 77.39 or 87.25% w/w or 1, 5, 4, and 8% w/v) were prepared to select the optimal excipients and excipient concentrations that can maintain the particle size distribution and mean particle size of AS01_{B}-adjuvanted OVA model vaccine subjected to TFFD followed by reconstitution.

Liquid AS01_{B} adjuvanted-OVA model vaccine was converted to dry powder using TFFD as described in Example 1. The frozen thin films were lyophilized using the same drying cycle mentioned in Example 1. The effect of repeated freezing and thawing on mean particle size of AS01_{B} adjuvanted-OVA model vaccine was investigated by exposing the dry powder prepared by TFFD as well as the liquid vaccine to three consecutive cycles of freezing at −20° C. for 8 h and thawing at 4° C. for 16 h. At the end of the third cycle, the mean particle size of the liquid and reconstituted powder was determined using DLS.

TABLE 1
Composition of various powder formulations prepared with varying
excipient and excipient concentration to maintain the particle size
distribution of AS01_{B}-adjuvanted OVA model vaccine after TFFD.

| | | Excipient | OVA | | PBS |
|---|---|---|---|---|---|
| | | concentration | (% | AS01_{B} | salts |
| Formulation | Excipient | (% w/w) | w/w) | (% w/w) | (% w/w) |
| ASOV-1 | Sucrose | 46.05 | 0.46 | 12.5 | 41 |
| ASOV-2 | Sucrose | 63.1 | 0.32 | 8.52 | 28.06 |
| ASOV-3 | Sucrose | 77.39 | 0.19 | 5.22 | 17.2 |
| ASOV-4 | Sucrose | 87.25 | 0.11 | 2.94 | 9.7 |
| ASOV-5 | Trehalose | 46.05 | 0.46 | 12.5 | 41 |
| ASOV-6 | Trehalose | 63.1 | 0.32 | 8.52 | 28.06 |
| ASOV-7 | Trehalose | 77.39 | 0.19 | 5.22 | 17.2 |
| ASOV-8 | Trehalose | 87.25 | 0.11 | 2.94 | 9.7 |
| ASOV-9 | Mannitol | 46.05 | 0.46 | 12.5 | 41 |
| ASOV-10 | Mannitol | 63.1 | 0.32 | 8.52 | 28.06 |
| ASOV-11 | Mannitol | 77.39 | 0.19 | 5.22 | 17.2 |
| ASOV-12 | Mannitol | 87.25 | 0.11 | 2.94 | 9.7 |

Liquid formulations were prepared in PBS (5 mM, pH 7.4) before being subjected to TFFD.
Note:
formulations were prepared by fixing OVA, AS01_{B} and PBS salt concentrations in the liquid formulation, while changing the concentration of the sugar or sugar alcohol (i.e., 1%, 2%, 4%, or 8%, w/v).

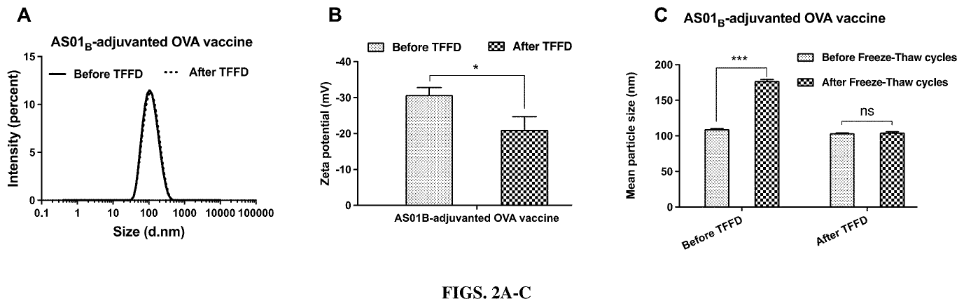

Results. As shown in FIG. 2A, the particle size distribution of AS01_{B}-adjuvanted OVA model vaccine was maintained after TFFD when sucrose was employed as an excipient at a concentration of 77.39% w/w (i.e., 4% w/v). However, the zeta potential values of the model vaccine significantly decreased after it was subjected to TFFD (p<0.05, FIG. 1B).

As shown in FIG. 2C, the mean particle size of AS01_{B}-adjuvanted OVA model vaccine did not significantly change after its dry powders was subjected to repeated freeze-and-thaw. On the other hand, subjecting the liquid model vaccine to repeated freeze-and-thaw caused significant aggregation (p<0.001).

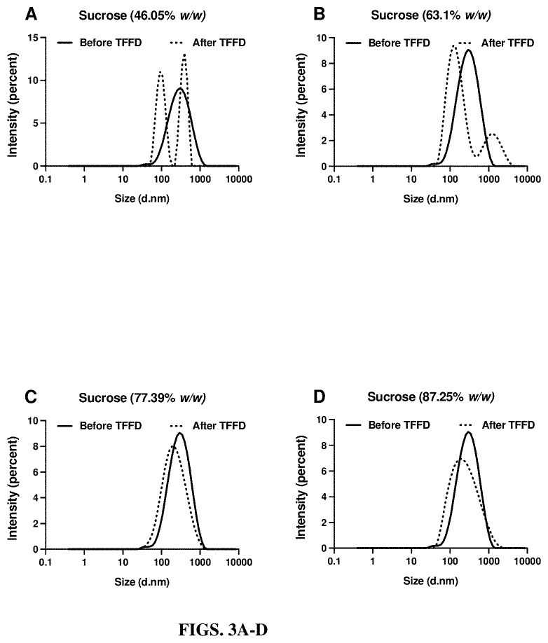

As shown in FIGS. 3A-B, low sucrose concentrations (i.e., 46.05 and 63.1% w/w, or 1% or 2%, w/v, in the liquid formulation) were ineffective in maintaining the particle size distribution of the model vaccine. On the other hand, sucrose concentrations above 77.39% (w/w, or 4%, w/v, in the liquid formulation) successfully maintained the particle size distribution of the AS01_{B}-adjuvanted vaccine after TFFD.

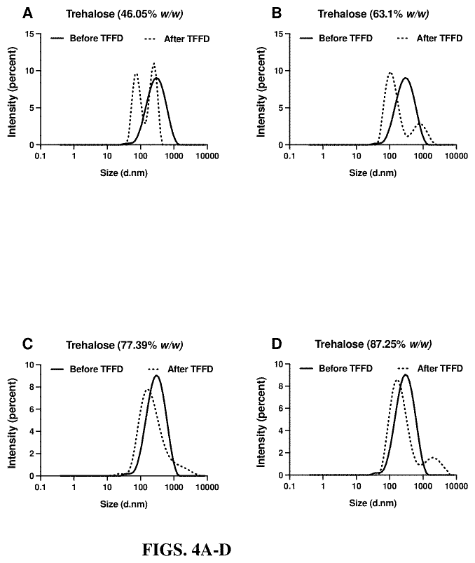

As shown in FIGS. 4A-B, low trehalose concentrations (i.e., 46.05 and 63.1% w/w, or 1% or 2%, w/v, in the liquid formulation) were ineffective in maintaining the particle size distribution of the model vaccine. The particle size distribution of the TFFD-processed model vaccine could be maintained at trehalose concentrations higher than 77.39% w/w (or 4%, w/v, in the liquid formulation); however, trehalose was not as effective as sucrose at the same concentration range (as compared to FIGS. 3C-D).

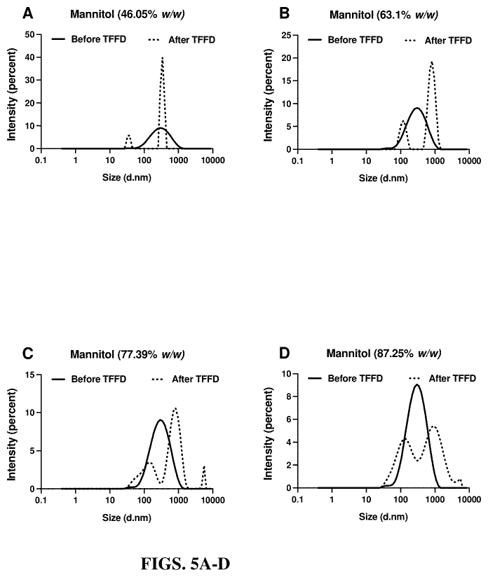

As shown in FIGS. 5A-D, mannitol was ineffective in maintaining the particle size distribution of AS01_{B}-adjuvanted OVA model vaccine at all the screened concentrations which could be attributed to mannitol crystallization.

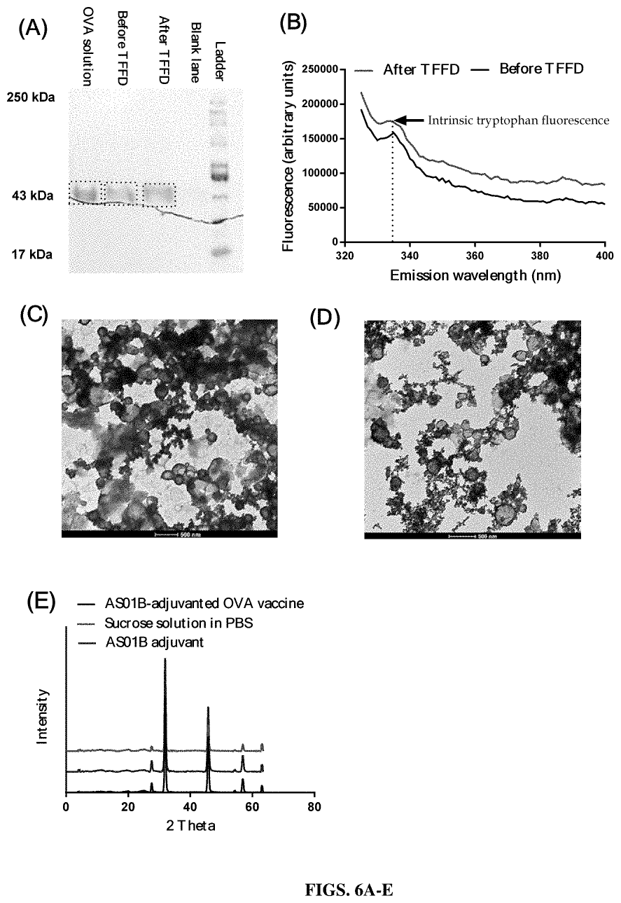

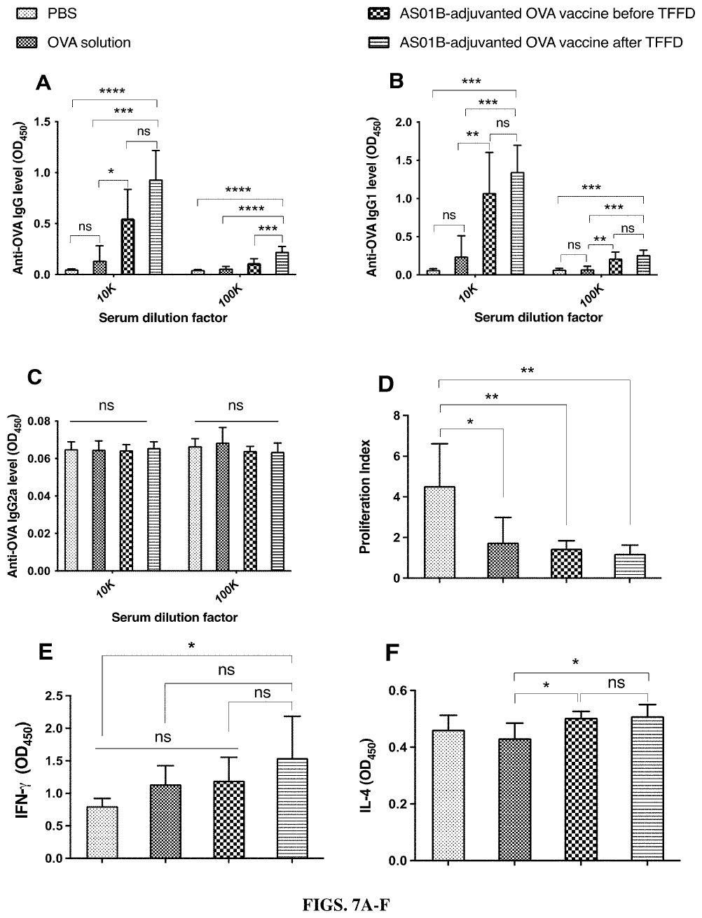

The integrity of OVA antigen after TFFD was evaluated using SDS-PAGE (FIG. 6A). No apparent aggregation or fragmentation of the OVA protein was detected after the model vaccine was subjected to TFFD and reconstitution. As shown in FIG. 6B, subjecting the vaccine to TFFD did not affect the wavelength of maximum tryptophan fluorescence emission (i.e., 335 nm); however, its fluorescence intensity was slightly higher, indicating that the secondary and tertiary structures of OVA were properly maintained after TFFD (Ghisaidoobe & Chung, 2014). As shown in FIGS. 6C-D, subjecting the vaccine to TFFD did not affect the morphology of liposomes, nor was it caused aggregations of the liposomes. As shown in FIG. 6E, all powders were amorphous and thus can help to better maintain the protein in its native structure as compared to in crystalline matrices. Finally, the moisture content of the powder determined using Karl Fischer titrator was determined to 4.76±0.25%. Prolonging the secondary drying during the sublimation step is expected to reduce the moisture content to lower levels, if needed. Importantly FIG. 7 shows that the immunogenicity of AS01_{B}-adjuvanted OVA vaccine was maintained after TFFD and reconstitution.

### Example 3—Dry Powder Compositions of SHINGRIX® Vaccine

Materials and Methods. The inventors tested SHINGRIX® vaccine (Zoster Vaccine Recombinant, Adjuvanted) from GlaxoSmithKline using single vial TFFD. The final product in the powder form was reconstituted, the zeta potential, particle size and size distribution of the reconstituted SHINGRIX® vaccine were measured.

SHINGRIX® vaccine was from GlaxoSmithKline. It was stored at 4° C. On the day of thin-film freezing, SHINGRIX® vaccine was prepared following the instruction on the FDA package insert. Briefly, the vaccine was prepared by reconstituting the lyophilized VZV glycoprotein E (gE) antigen component (lyophilized powder) with the accompanying AS01_{B} adjuvant suspension component (liquid). The reconstituted vaccine appeared as an opalescent and colorless liquid. Based on SHINGRIX® prescribing information, after reconstitution, each 0.5 mL dose contains 50 mcg of gE, 50 mcg of MPL, 50 mcg of QS-21, 20 mg of sucrose (or 4%, w/v), 4.385 mg of NaCl, 1 mg of DOPC, 0.54 mg of potassium dihydrogen phosphate, 0.25 mg of cholesterol, 0.16 mg of sodium dihydrogen phosphate dihydrate, 0.15 mg of disodium phosphate anhydrous, 0.116 mg of dipotassium phosphate, and 0.08 mg of polysorbate 80 (or 0.016% w/w).

To prepare the TFFD-processed powder, a salinized glass vial (adptiQ® R20 glass vial, Mainz, Germany) was immersed into liquid nitrogen to create a cryogenically cooled surface in the inner bottom of the vial. A syringe with a 21G1 needle was used to add 100 μL of the SHINGRIX® vaccine liquid dropwise to the bottom of the vial so that the droplets, when in contact with the surface, rapidly frozen into thin films. Then lyophilization was performed as described in Example 1. After lyophilization, vacuum was released, and nitrogen was filled inside the lyophilizer. The glass vials were capped tightly with rubber stoppers using the automatic stopper-recap function in the lyophilizer and sealed with aluminum cap. The vials were then stored at 4° C. for future use.

Vials with thin-film freeze-dried SHINGRIX® vaccine powder were reconstituted with water. Mean particle size, size distribution and zeta potential of the reconstituted SHINGRIX® vaccine, after 10-fold dilution to reach a total volume of 1 mL, were determined by DLS. The morphology of the SHINGRIX® vaccine was examined using an FEI Tecnai transmission electron microscope (TEM).

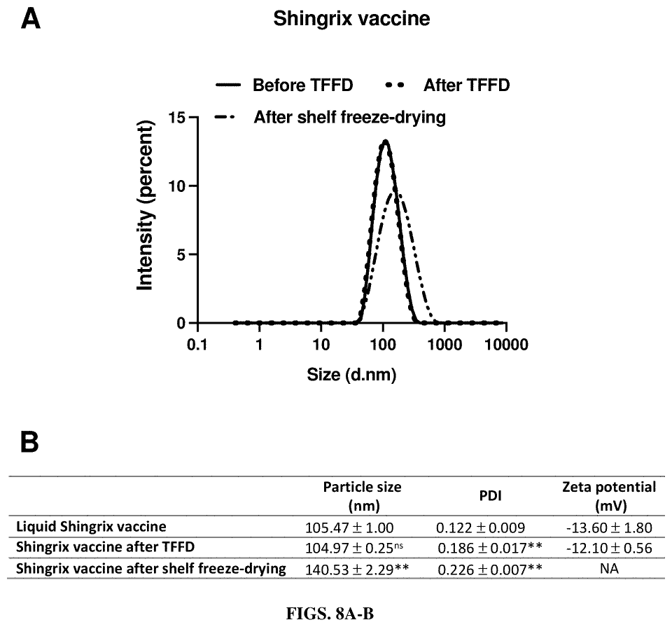

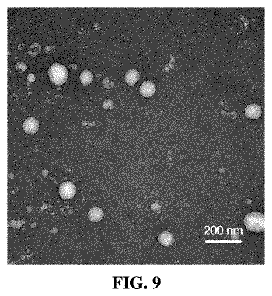

Results. As shown in FIGS. 8A-B and FIG. 9, subjecting the SHINGRIX® vaccine to TFFD and reconstitution did not cause significant particle aggregation, change of zeta potential or particle morphology. It is feasible to transform the commercially available AS01_{B}-adjuvanted SHINGRIX® vaccine that contains a lyophilized gE protein antigen vial and a vial that contains the AS01_{B} adjuvant in a liquid suspension to a single vial that contains the AS01_{B} adjuvant and gE antigen in a dry powder by TFFD without causing aggregation. However, subjecting the SHINGRIX® vaccine to conventional shelf freeze-drying and reconstitution cause an increase in the particle size (FIG. 8). From FIGS. 8A-B, and FIGS. 9A-D, the inventors conclude that subjecting the reconstituted liquid SHINGRIX® vaccine to TFFD and reconstitution did not result in particle aggregation but subjecting it to conventional shelf freeze-drying did.

### Example 4—Dry Powder Compositions of AS01_{B}-Adjuvanted Fluzone Quadrivalent® Vaccine

Materials and Methods. Fluzone Quadrivalent® is an FDA approved unadjuvanted, liquid vaccine for the prevention of influenza disease caused by influenza subtype A and subtype B viruses. It contains the hemagglutinin (HA) antigenic proteins of four influenza strains at a total concentration of 60 μg HA/0.5 mL. Liquid AS01_{B}-adjuvanted Fluzone Quadrivalent® vaccine was prepared by adding liquid 0.5 mL of AS01_{B} adjuvant to 0.5 mL of liquid Fluzone Quadrivalent®. The adjuvanted vaccine was then converted to dry powder using TFFD and lyophilization as described in the previously mentioned examples. Dry powders of AS01_{B}-adjuvanted Fluzone Quadrivalent® vaccine was then reconstituted in water and characterized according to particle size distribution, zeta potential, SDS-PAGE analysis, intrinsic tryptophan fluorescence, and hemagglutination assay. The effect of repeated freezing and thawing on the particle size distribution of liquid and dry powder forms of AS01_{B}-adjuvanted Fluzone Quadrivalent® vaccine was also investigated.

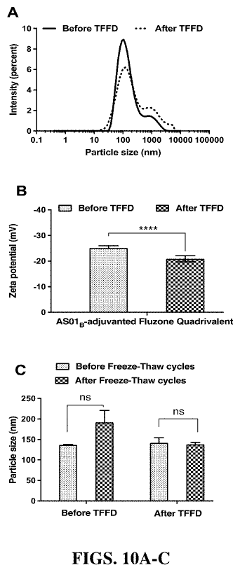

Results. As shown in FIG. 10A, the particle size distribution profile of the AS01_{B}-adjuvanted Fluzone Quadrivalent® vaccine was generally preserved after the vaccine was subjected to TFFD and reconstitution, while the zeta potential value was significantly reduced (FIG. 10B) (p<0.001). As shown in FIGS. 10C-D, overall, the dry powder successfully maintained the mean particle size distribution of the vaccine after it was exposed to repeated freeze-and-thaw (FIG. 10C), but the mean particle size distribution of the liquid vaccine increased after it was subjected to repeated freeze-and-thaw (FIG. 9D, see the liposome peak at around 100 nm shifted to 200-300 nm, ****p<0.001).

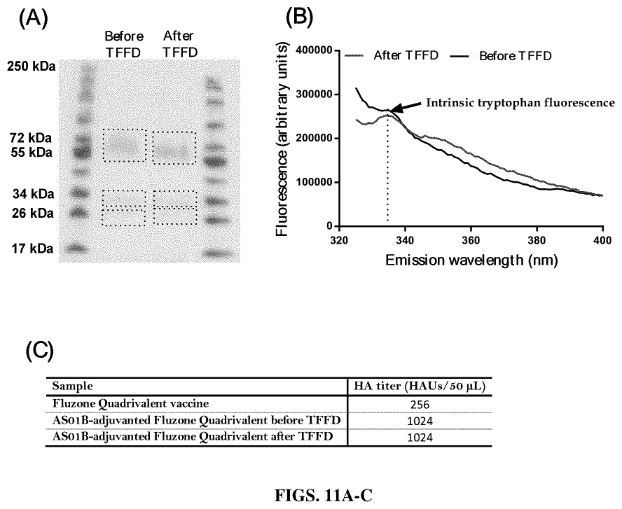

As shown in FIGS. 11A-B, SDS-PAGE revealed no TFFD-induced protein fragmentation or aggregation. The intensity of tryptophan fluorescence was slightly altered, and the wavelength of maximum tryptophan fluorescence did not shift. Thus, TFFD likely have maintained the primary and tertiary structures of various vaccine antigens. As shown in FIG. 11C, the HA titer of the AS01_{B}-adjuvanted Fluzone Quadrivalent® did not change after it was subjected to TFFD and reconstitution. Overall, it is concluded that AS01_{B}-adjuvanted Fluzone Quadrivalent vaccine was successfully transformed into a dry powder by TFFD without affecting the integrity and potency/functionality of the antigens.

Conclusion. Using three vaccines (i.e., AS01_{B}-adjuvanted OVA model vaccine, AS01_{B}-adjuvanted Fluzone Quadrivalent® vaccine, and SHINGRIX® (AS01_{B}-adjuvanted varicella zoster virus glycoprotein)), the inventors provide evidence supporting the feasibility of transforming AS01_{B}-adjuvanted vaccines from a liquid suspension to a dry powder by thin-film freeze-drying, while preserving the particle size distribution, antigen integrity, as well as potency and/or immunogenicity of the vaccines upon reconstitution. Moreover, the thin-film freeze-dried AS01_{B}-adjuvanted vaccines were not sensitive to repeating slow freezing.

### Example 5—Inhalable Dry Powder Compositions of AS01_{B}-Adjuvanted OVA Model Vaccine

Background. In a cross-sectional survey, needle fear was the primary reason of vaccination non-compliance for 7% of children and 8% of parents attending a public museum in Toronto, Canada (Taddio et al., 2012). A meta-analysis showed that one in six healthcare workers in long-term care facilities did not receive influenza vaccination due to needle phobia (McLenon & Rogers, 2019). Moreover, needle phobia among adults may be a challenge towards mass vaccination against SARS-CoV-2 pandemic (Love & Love, 2021). On the other hand, mucosal vaccine delivery (e.g., pulmonary and nasal delivery) is needle-free and can achieve high patient compliance (Zhang et al., 2021). Dendritic cells are present in the tissue microenvironments of the lower and upper respiratory tracts in particular within the epithelium lining of the conducive airways. Dendritic cells have been also identified on the alveolar surface (Holt et al., 1999). Alveolar macrophages are also abundant in the periphery and interstitium of the lungs (Lu & Hickey, 2007). Therefore, mucosal immunization via the respiratory tract can elicit both localized and systemic immune responses that may be required to provide long-term protection against some pathogens (e.g., filoviruses and coronaviruses) (Bajrovic et al., 2020)(Cross et al., 2018)(Mudgal et al., 2020)(Heida et al., 2021).

The advantages of liposomes for pulmonary vaccine delivery are well-recognized. Furthermore, available data shows the safety and tolerability of inhalable liposomal antibiotics (Marasini & Kaminskas, 2019)(Bassetti et al., 2020). However, current vaccine pre-clinical studies are mainly focused on intranasal liposomal vaccine delivery (Marasini & Kaminskas, 2019)(Bassetti et al., 2020). This could be attributed to the detrimental effect of nebulization on liposome membrane integrity that results in premature antigen release (Marasini & Kaminskas, 2019)(Bassetti et al., 2020). Ultimately, liposomal dry powders for inhalation can avoid nebulization-induced stress of liposomal membrane. Additionally, inhalable dry powders have superior stability profiles and dry powder inhaler (DPI) devices are easy to use (Qiu et al., 2019). Although the development of liposomal dry powders has been widely investigated using various powder engineering technologies (e.g., freeze-drying, spray-drying, and spray freeze-drying), the development of inhalable dry powders for AS01_{B}-adjuvanted vaccines has not yet been investigated. The previously developed inhalable liposomal powders showed fine particle fraction (FPF) values between 30% and 50% for spray-dried powders (Ourique et al., 2014)(Manca et al., 2014)(Yu et al., 2020a), between 17% and 30% for conventional shelf freeze-dried powders (Shah & Misra, 2004a)(Shah & Misra, 2004b), and between 34 and 50% for spray freeze-dried or ultrasonic spray freeze-dried powders (Yu et al., 2020b)(Ye et al., 2017).

Materials and Methods. Liquid formulation of AS01_{B}-adjuvanted OVA model vaccine containing sucrose as a stabilizer was diluted two-fold in water. Then, the liquid vaccine formulation was subjected to TFFD as described in Example 1. The aerosol performance of the powder was determined using a Next Generation Impactor (NGI, MSP Corp, Shoreview, Minn.) connected to a High-Capacity Pump HCP5 (Copley Scientific, Nottingham, UK) and a Critical Flow Controller TPK 2100-R (Copley Scientific) as described before (Sahakijpijarn et al., 2020). The dry powder (1-3 mg) was loaded into a size 3 hydroxypropyl methylcellulose capsule (VCaps plus, Lonza, Inc., Morristwon, N.J.), which was then loaded into a high resistance Plastiape® RS00 DPI device (Plastiape S.p.A, Osnago, Italy). The NGI was run for 4 s per actuation at a controlled flow rate of 60 L/min. The dry powder deposited on various stages of the NGI, the adapter, the DPI device, the induction port and as well as those remained in the capsule was collected in water. Sucrose content was determined using an HPLC system (1220 Infinity II HPLC, Agilent, Santa Clara, Calif.) as previously reported (Hufnagel et al., 2022). Copley Inhaler Testing Data Analysis Software (CITDAS) Version 3.10 (Copley Scientific) was used to calculate the mass median aerodynamic diameter (MMAD), the geometric standard deviation (GSD), the FPF of delivered and recovered doses, and the emitted fraction (EF) (Sahakijpijarn et al., 2020)(Meenach et al., 2013).

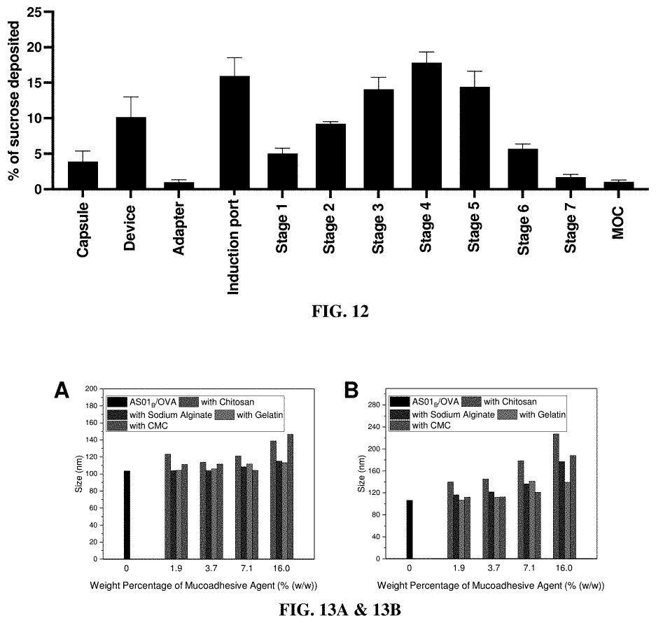

Results. In this study, the applicability of using TFFD to formulate inhalable dry powders of AS01_{B}-adjuvanted OVA vaccine containing sucrose as a stabilizer at lipid to sugar ratio of 1:16 w/w was further investigated. FIG. 12 shows the aerosol performance of the vaccine dry powders determined using an NGI and a high resistance Plastiape® RS00 DPI. Aerosol deposition of TFFD-processed vaccine powder on impaction stage 2 all the way to impaction stage 7 was observed. The dry powder formulation showed an MMAD of 2.4±0.1 μm and a FPF (of delivered dose) >66% (Table 2), indicating that it can reach deep lung upon oral inhalation. The good aerosol performance of dry powder formulations prepared using TFFD can be attributed to the brittle matrix nature of the powders produced by the TFF process. DPIs can shear the brittle matrix powders into respirable, low-density particles having high tendency for deep lung deposition (Sahakijpijarn et al., 2020). Pulmonary immunization can enhance cross-presentation and Th-1-biased immunity, which are essential for protection against viral infections (Marasini & Kaminskas, 2019). The inhalable dry powders of AS01_{B}-adjuvanted vaccines prepared using the TFFD technology may help provide new mucosal adjuvants and vaccine formulations for efficient pulmonary vaccination. The AS01_{B}-adjuvanted vaccine composition may also be adjusted before TFFD to generate a dry powder for intranasal immunization.

TABLE 2
The aerosol properties of TFFD-processed AS01_{B}-adjuvanted
OVA model vaccine dry powder prepared using TFFD. Data
are mean ± SD (n = 3).

| | | | FPF_{<5 μm} | FPF_{<5 μm} | EF |
|---|---|---|---|---|---|
| | | | (%, | (%, | (%, |
| Drying | MMAD | | Delivered | Recovered | Recovered |
| technology | (μm) | GSD | Dose) | Dose) | Dose) |
| TFFD | 2.4 ± 0.1 | 2.3 ± 0.1 | 66.3 ± 4.9 | 57.1 ± 6.4 | 85.9 ± 3.6 |

### Example 6: Preparation of AS01_{B}-Adjuvanted Vaccine Powers with a Mucoadhesive Agent

Introduction Data from previous studies have demonstrated that delivering vaccines by the intranasal route could induce systematic and mucosal immune responses (Birkhoff et al., 2009). For potential intranasal delivery of the dry thin film of the AS01_{B}-adjuvanted vaccine, the inventors tested adding mucoadhesive agents into the thin film adjuvant composition to increase the residence time of the vaccine in the nasal cavity upon intranasal administration. Mucoadhesive agents tested include chitosan, sodium alginate, gelatin, and sodium carboxymethylcellulose (CMC). Each of the mucoadhesive agents has its own unique mechanism(s) of interaction with the nasal mucosal surface (Sogias et al., 2008)(Kesavan et al., 2010)(Grabovac et al., 2005)(Dekina et al., 2016). A challenge is that the mucoadhesive agents could interact with the AS01_{B}/OVA vaccine and change the structure of the AS01_{B} and/or the vaccine candidate, and thus affecting the efficacy of the AS01_{B}/OVA vaccine. Therefore, the inventors studied the effect of the mucoadhesive agents and their concentrations on the AS01_{B}/OVA vaccine before and after being subjected to TFFD.

Materials. Chitosan (medium molecular weight), sodium alginate, gelatin, sodium carboxymethylcellulose (CMC), and porcine mucin type III were from Sigma-Aldrich. Dulbecco's phosphate-buffered saline (DPBS) was from Gibco.

Preparation of the AS01_{B}/OVA model vaccine. The AS01_{B}/OVA model vaccine was prepared as aforementioned. The liposome dispersion contained 4.0 mg of DOPC, 1.0 mg of cholesterol, and 0.2 mg of MPL in 0.5 mL of DPBS.

Preparation of AS01_{B}/OVA model vaccine with different mucoadhesive agents. To prepare the AS01_{B}/OVA model vaccine with different concentrations of chitosan, sodium alginate, gelatin, or CMC, 50 μg of QS-21, 50 μg of OVA, and 19.5 mg of sucrose were added to 125 μL of the liposome dispersion. The stock solution of chitosan was prepared by dissolving chitosan 2% (w/v) in 0.1 M acetic acid aqueous solution. The stock solutions of sodium alginate, alginate, and CMC were prepared by dissolving them (2% w/v) in DPBS. Different volumes of the mucoadhesive agent-containing solutions were then added to the concentrated AS01_{B}/OVA vaccine to achieve a final concentration of 0.1%, 0.2%, 0.4%, or 1% w/v, corresponding to 1.9%, 3.7%, 7.1%, or 16.0% w/w of the mucoadhesive agent in the theoretical total weight of all components, except water. The final volume of the AS01_{B}/OVA with different concentrations of mucoadhesive agents was then adjusted to 0.5 mL with DPBS. The vaccine preparations with different concentrations of mucoadhesive agents were converted into dry powders by TFFD as mentioned above. The particle size of the vaccines before and after being subjected to TFFD and reconstitution (with water) was measured by dynamic light scattering (DLS) using a Malvern Nano ZS. Samples were diluted 50 times with PBS before the measurement. The pH value of all the samples was approximately 7.4.

Evaluating the integrity of the OVA. SDS-PAGE was applied to evaluate the integrity of OVA in AS01_{B}/OVA model vaccine compositions with 0%, 1.9%, or 3.7% of CMC before and after TFFD. Vaccine powders were reconstituted with water. The samples were mixed with Laemmli sample buffer 4× containing 10% 2-mercaptoethanol and boiled for 10 min at 100° C. Finally, 30 μL of each sample were loaded into the wells of a 4-20% precast polyacrylamide gel (Bio-Rad). The electrophoresis was performed at 90 V for 90 min. The SDS-PAGE gel was stained with Coomassie G-250, and the image was captured with a camera.

In vitro mucoadhesion test of the TFFD vaccine powders. The procedure of the in vitro mucoadhesion test was adopted from a method published by Trenkel et al. (Trenkel & Scherließ, 2021). To simulate the human nasal mucosal surface, 10-cm Petri dishes were coated with either 1.5% w/v agar in DPBS (pH=6) or 1.5% w/v agar plus 2% w/v porcine mucin in DPBS (pH=6). The solutions may require microwave heating to make agar and porcine mucin completely dissolve. The coating layer was solidified by incubating for 2 h at room temperature and then 30 min at 4° C. After solidification, the coated dishes were incubated in a 32° C. incubator-shaker (Fisher Scientific) until equilibrium. And approximately 5 mg of TFFD vaccine powders were placed onto the gel. The dishes were then turned vertically and incubated for 2 h at 32° C. The maximum movement of the TFFD vaccine powders at the time points 10 min, 20 min, 30 min, 1 h, and 2 h were recorded.

Results: Screening of the potential mucoadhesive agents. To increase the residence time of the vaccine in the nasal cavity, different mucoadhesive agents, chitosan, sodium alginate, gelatin, or CMC, were added to the AS01_{B}/OVA vaccine. The final concentrations of the mucoadhesive agents tested in the powders ranged from 1.9% to 16% by weight. FIG. 13A shows the particle sizes of the AS01_{B}/OVA vaccine containing different concentrations of the mucoadhesive agents before being subjected to TFFD. Sodium alginate, gelatin, and CMC did not significantly affect the particle size of AS01_{B}/OVA vaccine, while chitosan caused a significant particle size increase, even when the concentration was only 1.9% by weight. Moreover, chitosan and CMC caused a significant particle size increase at 16.0% by weight (FIG. 13A).

After TFFD, only the vaccine formulations contained gelatin or CMC at 1.9% or 3.7% by weight maintained their particle size upon reconstitution (FIG. 13B). The AS01_{B}/OVA vaccine formulations with CMC at 1.9% or 3.7%, w/w, were selected for further analysis.

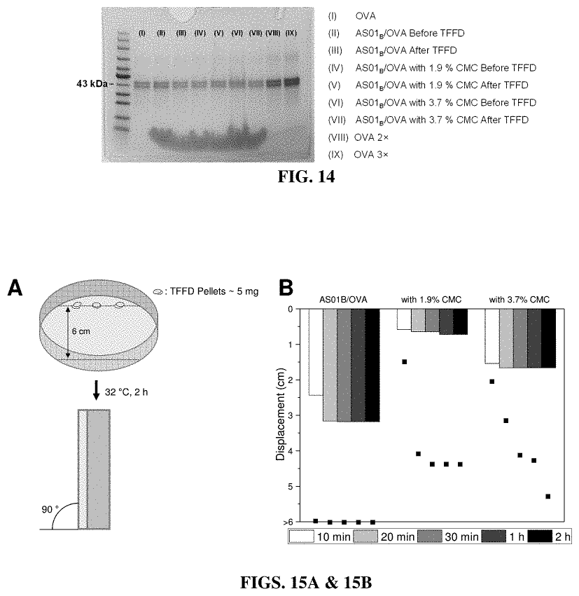

The integrity of the OVA In the AS01_{B}/OVA vaccines before and after being subjected to TFFD To evaluate the impact of the TFFD process on the integrity of OVA, SDS-PAGE was used. The SDS-PAGE result of the standard OVA solutions showed two bands of around 43 kDa (FIG. 14), which might be attributed to the different glycosylated forms of OVA. A comparison of the SDS-PAGE results of the AS01_{B}/OVA vaccine with different concentrations of CMC before and after TFFD showed that the integrity of OVA was maintained after TFFD (FIG. 14).

In vitro mucoadhesion test of the AS01_{B}/OVA powders. The petri dishes coated with 1.5% agar or 1.5% agar with 2% porcine mucin were used to simulate nasal mucosal surface. In the in vitro mucoadhesion test, the TFFD pellets of the AS01_{B}/OVA powders prepared with 0%, 1.9%, or 3.7% CMC were placed on the coated Petri dish surface. The dishes were turned vertically and incubated at 32° C. (FIG. 15A). The TFFD pellets absorbed moisture from the gel or the atmosphere and flowed down slowly to the bottom (FIG. 15A). The maximum traveling distances of the powders at different time points are shown in FIG. 15B. With the gel containing 1.5% agar only, the displacement of the AS01_{B}/OVA almost reached 6 cm after 10 min of incubation, and the displacements of AS01_{B}/OVA powders with 1.9% and 3.7% CMC were only 1.5 cm and 2 cm, respectively. The displacements of AS01_{B}/OVA powders with 1.9% and 3.7% CMC both reached 4.5 cm after 1 h of incubation, indicating the adhesive property of CMC-containing AS01_{B}/OVA vaccine powders.

With the gel containing 1.5% agar and 2% porcine mucin, the displacements of the three groups all decreased significantly, as compared to that with the gel containing 1.5% agar only (FIG. 15B). However, the displacement of the AS01_{B}/OVA powder was still larger than that of AS01_{B}/OVA powders with 1.9% or 3.7% of CMC. The displacement of the AS01_{B}/OVA with 1.9% CMC was slightly smaller than that with 3.7% CMC (FIG. 15B). Taken together, it was concluded that the AS01_{B}/OVA vaccine powders prepared with CMC were mucoadhesive.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### V. References

The following references, to the extent that they provide exemplary procedural or other details supplementary to those set forth herein, are specifically incorporated herein by reference.
Bajrovic et al., “Novel technology for storage and distribution of live vaccines and other biological medicines at ambient temperature,” Sci Adv, 2020. 6(10).
Bassetti et al., “Inhaled Liposomal Antimicrobial Delivery in Lung Infections,” Drugs, 2020. 80(13): p. 1309-1318.
Birkhoff et al., “Advantages of Intranasal Vaccination and Considerations on Device Selection,” Indian J Pharm Sci 71 (2009) 729-31.
Chen et al., “Improving the reach of vaccines to low-resource regions, with a needle-free vaccine delivery device and long-term thermostabilization,” J Control Release, 152 (2011) 349-355.
Cross et al., “Post-exposure treatments for Ebola and Marburg virus infections,” Nat Rev Drug Discov, 2018. 17(6): p. 413-434.
Dekina et al., “Gelatin/carboxymethyl cellulose mucoadhesive films with lysozyme: Development and characterization,” Carbohydr Polym, 147, 2016, 208-215.
Di Pasquale et al., “Vaccine Adjuvants: from 1920 to 2015 and Beyond,” Vaccines (Basel), 3 (2015) 320-343.
Didierlaurent et al., “Adjuvant system AS01: helping to overcome the challenges of modern vaccines,” Expert Rev Vaccines, 16 (2017) 55-63.
Ghisaidoobe & Chung, “Intrinsic tryptophan fluorescence in the detection and analysis of proteins: a focus on Förster resonance energy transfer techniques,” Int J Mol Sci, 15 (2014) 22518-22538.
Grabovac et al., “Comparison of the mucoadhesive properties of various polymers,” Adv Drug Deliv Rev 57(2005) 1713-23.
Heida et al., “Inhaled vaccine delivery in the combat against respiratory viruses: a 2021 overview of recent developments and implications for COVID-19,” Expert Rev Vaccines, 2021: p. 1-18.
Holt et al., “Functional studies on dendritic cells in the respiratory tract and related mucosal tissues,” J Leukoc Biol, 1999. 66(2): p. 272-5.
Hufnagel et al., “Dry powders for inhalation containing monoclonal antibodies made by thin-film freeze-drying.” Int J Pharm, 2022. 618: p. 121637.
Kesavan et al., “Sodium alginate based mucoadhesive system for gatifloxacin and its in vitro antibacterial activity,” Sci Pharm 78 (2010) 941-57.
Li et al., “A method of lyophilizing vaccines containing aluminum salts into a dry powder without causing particle aggregation or decreasing the immunogenicity following reconstitution,” J Control Release, 204 (2015) 38-50.
Love & Love, “Considering Needle Phobia among Adult Patients During Mass COVID-19 Vaccinations,” J Prim Care Community Health, 2021. 12: p. 21501327211007393.
Lu & Hickey, “Pulmonary vaccine delivery,” Expert Rev Vaccines, 2007. 6(2): p. 213-26.
Manca et al., “Fabrication of polyelectrolyte multilayered vesicles as inhalable dry powder for lung administration of rifampicin,” Int J Pharm, 2014. 472(1-2): p. 102-9.
Mandon et al., “Novel calixarene-based surfactant enables low dose split inactivated vaccine protection against influenza infection,” Vaccine, 38 (2020) 278-287. Marasini & Kaminskas, “Subunit-based mucosal vaccine delivery systems for pulmonary delivery—Are they feasible?” Drug Dev Ind Pharm, 2019.45(6): p. 882-894.
Matthias et al., “Freezing temperatures in the vaccine cold chain: a systematic literature review,” Vaccine, 25 (2007) 3980-3986.
McLenon & Rogers, “The fear of needles: A systematic review and meta-analysis,” J Adv Nurs, 2019. 75(1): p. 30-42.
Meenach et al., “Characterization and aerosol dispersion performance of advanced spray-dried chemotherapeutic PEGylated phospholipid particles for dry powder inhalation delivery in lung cancer,” Eur J Pharm Sci, 2013. 49(4): p. 699-711.
Mudgal et al., “Prospects for mucosal vaccine: shutting the door on SARS-CoV-2,” Hum Vaccin Immunother, 2020. 16(12): p. 2921-2931.
Ourique et al., “Redispersible liposomal-N-acetylcysteine powder for pulmonary administration: development, in vitro characterization and antioxidant activity,” Eur J Pharm Sci, 2014. 65: p. 174-82.
Qiu et al., “Effective mRNA pulmonary delivery by dry powder formulation of PEGylated synthetic KL4 peptide,” J Control Release, 2019. 314: p. 102-115.
Sahakijpijarn et al., “Development of Remdesivir as a Dry Powder for Inhalation by Thin Film Freezing,” Pharmaceutics, 2020. 12(11).
Shah & Misra, “Development of liposomal amphotericin B dry powder inhaler formulation,” Drug Deliv, 2004a. 11(4): p. 247-53.
Shah & Misra, “Liposomal amikacin dry powder inhaler: effect of fines on in vitro performance,” AAPS PharmSciTech, 2004b. 5(4): p. e65.
Sogias et al., “Why is chitosan mucoadhesive?” Biomacromolecules 9 (2008) 1837-42.
Taddio et al., “Survey of the prevalence of immunization non-compliance due to needle fears in children and adults,” Vaccine, 2012. 30(32): p. 4807-12.
Trenkel & Scherließ, “Nasal Powder Formulations: In-Vitro Characterisation of the Impact of Powders on Nasal Residence Time and Sensory Effects,” Pharmaceutics, 13, 2021.
Vandepapelière et al., “Vaccine adjuvant systems containing monophosphoryl lipid A and QS21 induce strong and persistent humoral and T cell responses against hepatitis B surface antigen in healthy adult volunteers,” Vaccine, 26 (2008) 1375-1386.
Yu et al., “Inhalable liposomal powder formulations for co-delivery of synergistic ciprofloxacin and colistin against multi-drug resistant gram-negative lung infections,” Int J Pharm, 2020a. 575: p. 118915.
Yu et al., “Optimization of inhalable liposomal powder formulations and evaluation of their in vitro drug delivery behavior in Calu-3 human lung epithelial cells,” Int J Pharm, 2020b. 586: p. 119570.
Ye et al., “Inhalable clarithromycin liposomal dry powders using ultrasonic spray freeze drying,” Powder Technology, 2017.305: p. 63-70.
Zhang et al., “Novel formulations and drug delivery systems to administer biological solids,” Adv Drug Deliv Rev, 2021. 172: p. 183-210.

1-15. (canceled)

16. A method for preparing an adjuvant thin film comprising:
applying a liquid adjuvant composition to a freezing surface, wherein said liquid adjuvant composition comprises monophosphoryl lipid A and/or saponin fraction QS-21, and a sugar or a sugar alcohol; and
allowing said liquid adjuvant composition to disperse and freeze on said freezing surface thereby forming an adjuvant thin film.

17. The method of claim 16, wherein said liquid adjuvant composition further comprises an antigen, such as an antigen in a subunit vaccine.

18. The method of claim 16, wherein said liquid adjuvant composition comprises dioleoyl phosphatidylcholine and cholesterol.

19. The method of claim 16, wherein said adjuvant thin film has a particle size distribution within about 10-50% or about 30% of the range of the liquid adjuvant composition.

20. The method of claim 16, wherein said sugar or sugar alcohol is present at about 40% to about 90% w/w or about 70% w/w.

21. The method of claim 17, wherein said antigen is varicella zoster virus glycoprotein E or comprises one, two, three or four distinct influenza hemagglutinin antigens.

22. The method of claim 16, wherein said adjuvant vaccine thin film comprises less than about 5% water.

23. The method of claim 16, wherein said adjuvant thin film further comprises an excipient.

24. The method of claim 23, wherein said excipient is a salt, a buffer, a detergent, a polymer, an amino acid, a second sugar or a preservative.

25. The method of claim 24, wherein said excipient is disodium edetate, sodium chloride, sodium citrate, sodium succinate, sodium hydroxide, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco's modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer.

26. The method of claim 23, comprising from about 10% to about 40% w/w of said excipient.

27. The method of claim 16, wherein said liquid adjuvant composition is exposed to said freezing surface from about 50 milliseconds to about 5 seconds.

28. The method of claim 16, wherein exposure comprises spraying or dripping droplets of said liquid adjuvant composition.

29. The method of claim 28, wherein the freezing surface temperature is about −180° C. to about 0° C., the diameters of the droplets are about 2-5 millimeters, and the droplets are dropped from a distance about 2 cm to 10 cm from the freezing surface.

30. The method of claim 27, further comprising contacting the droplets with a freezing surface having a temperature differential of at least about 30° C. between the droplets and the surface and or wherein the freezing rate of said droplets is between about 10 K/second and about 10³ K/second.

31. The method of claim 16, further comprising removing the solvent from the vaccine thin film to form a dry adjuvant composition, optionally by lyophilization.

32. (canceled)

33. The method of claim 16, wherein the thin adjuvant film is formulated as an aerosol for inhalation in the lungs or the nasal cavity.

34-41. (canceled)

42. The method of claim 16, wherein said liquid adjuvant composition comprises one or more of a neutral lipid, a cationic lipid, an amphipathic lipid and/or an anionic lipid.

43. The method of claim 16, wherein said liquid adjuvant composition comprises a lipid selected from the group consisting of soya lecithin, cholesterol, Soya phosphatidylcholine, hydrogenated soybean phosphatidylcholine, 1,2-Distearoyl-sn-glycero-3-phosphoglycerol, phosphatidylcholine, dipalmitoylphosphatidylcholine, egg phosphatidylcholine, 1,2-diphytanoyl-sn-glycero-3-phosphocholine, 1,2-Dioleoyl-sn-glycero-3-phospho-rac-(1-glycerol), 1,2-dioleoyl-3-trimethylammonium-propane, 1,2-Dioleoyl-sn-glycero-3-phosphocholine, 1,2-dipalmitoyl-sn-glycero-3-phosphocholine, 1-paltnitoyl-2-lyso-sn-gycero-3-pllosplloclloline, 1-palmitoyl-2-oleoyl-sn-glycero-3-ethylphosphocholine, 1,2-dimyristoyl-sn-glycero-3-phosphocholine, 1-palmitoyl-2-oleoyl-sn-glycero-3-phosphocholine, and 1,2-dioleoyl-sn-glycero-3-phosphoethanolamine.

44. (canceled)

45. The method of claim 16, wherein said liquid adjuvant composition comprises a pharmacological active ingredient.

46-52. (canceled)
